(19) United States
(12) Patent Application Publication
(10) Pub. No.: US 2003/0087253 A1
(43) Pub. Date: May 8, 2003

(54) Polynucleotide markers for ovarian cancer

(76) Inventors: Chao Zhang, Moraga, CA (US); Behzad Mahini, Saratoga, CA (US); Randi E. Krasnow, Stanford, CA (US); Michael G. Walker, Sunnyvale, CA (US)

Correspondence Address: INCYTE GENOMICS, INC., 3160 PORTER DRIVE, PALO ALTO, CA, 94304, US

(21) Appl. No.: 10/113,234

(22) Filed: Mar. 28, 2002

Related U.S. Application Data
(60) Provisional application No. 60/280,520, filed on Mar. 30, 2001.

(51) Int. Cl. C12Q 1/68; G01N 33/574; C07K 14/72; C12P 21/02; C12N 5/06; C07H 21/04

(52) U.S. Cl. 435/6; 435/7.23; 435/69.1; 435/320.1; 435/325; 530/350; 536/23.5

(57) ABSTRACT

The invention provides polynucleotides that are specifically and differentially expressed in ovarian cancer, particularly serous papillary carcinoma. The invention also provides compositions, probes, expression vectors, host cells, proteins encoded by the polynucleotides and antibodies which specifically bind the proteins. The invention also provides methods for the diagnosis, prognosis and treatment of ovarian cancer.

### FIELD OF THE INVENTION

The invention relates to polynucleotides which are useful for the diagnosis, prognosis and treatment of ovarian cancer, particularly serous papillary carcinoma.

### BACKGROUND OF THE INVENTION

Ovarian cancer is the leading cause of death from gynecologic malignancy and the fourth leading cause of cancer death among American women. Since ovarian tumors produce few early signs, the disease is often not identified until its later stages (stage III or IV). About one in 70 women eventually develops ovarian cancer, and one in 100 women dies of it. Confirmed metastasis of papillary serous carcinoma is associated with a survival of approximately one year.

Ovarian cancer affects predominantly perimenopausal and postmenopausal women, and incidence of the disease is higher in industrialized countries with a higher dietary fat intake. Familial predisposition to endometrial, breast, or colon cancer increases risk as does nulliparity, infertility, late-childbearing, and delayed menopause; however, the use of oral contraceptives significantly decreases risk (The Merck Manual, 1992, Rahway N.J., Sec 14, Ch 171, pp 1827-1829).

Primary epithelial tumors make up 90% of ovarian cancers and include serous papillary carcinoma, also known as serous cystadenocarcinoma, mucinous cystadenocarcinoma, and endometrioid and mesonephric malignancies. Serous papillary carcinomas account for 50% of primary epithelial ovarian cancers.

To date ultrasonography is the method of choice for identification of stage I ovarian cancer, but it is only effective where familial factors, abdominal symptoms, or abnormalities found during routine pap smears raise the need for further examination (Karlan et al. (1999) Am J Obstet Gynecol 180:917-28; Jimenez-Ayala et al. (1996) Acta Cytol 40:765-9). Ovarectomy is the treatment of choice, and peritoneal washing cytology during surgery has been found to be a useful prognostically (Suzuki et al. (1999) Oncol Rep 6:1009-12).

Since there is only one non-invasive test that women can obtain which will point out the onset of this silent killer, the identification of diagnostic and prognostic markers for ovarian cancer satisfies a need in the art. The present invention provides polynucleotides which are useful in the diagnosis, prognosis, and treatment of individuals with ovarian cancer, particularly serous papillary carcinoma.

### SUMMARY OF THE INVENTION

The invention provides a combination comprising a plurality of polynucleotides having the nucleic acid sequences of SEQ ID NOs: 1-9 that are specifically and differentially expressed in ovarian cancer or the complements of SEQ ID NOs: 1-9. The invention also provides an isolated polynucleotide having a nucleic acid sequence selected from SEQ ID NOs: 1-9 and the complements thereof. In different aspects, each polynucleotide is used as a diagnostic, as a probe, in an expression vector, and in the prognosis and treatment of ovarian cancer.

The invention provides a method of using a combination comprising a plurality of polynucleotides or an isolated polynucleotide to screen a plurality of molecules to identify at least one ligand which specifically binds a polynucleotide, the method comprising contacting the combination or the polynucleotide with molecules under conditions to allow specific binding; and detecting specific binding, thereby identifying a ligand which specifically binds the polynucleotide. In one embodiment, the molecules are selected from DNA molecules, RNA molecules, peptide nucleic acids, peptides, and proteins. The invention further provides a method for using a combination comprising a plurality of polynucleotides or an isolated polynucleotide to detect expression in a sample containing nucleic acids, the method comprising hybridizing the combination or polynucleotide to the nucleic acids under conditions for formation of one or more hybridization complexes; and detecting hybridization complex formation, wherein complex formation indicates expression in the sample. In one embodiment, the combination or polynucleotide is attached to a substrate. In another embodiment, the sample is from kidney. In yet another embodiment, the nucleic acids are amplified prior to hybridization. In still another embodiment, complex formation is compared to standards and is diagnostic of ovarian cancer including, but not limited to, any tumor of the ovary of primary epithelial origin and specifically serous papillary carcinoma (also known as serous cystadenocarcinoma), mucinous cystadenocarcinoma, endometrioid and mesonephric malignancies, ovarian adenocarcinomas, and borderline ovarian carcinomas.

The invention provides a vector containing the polynucleotide, a host cell containing a vector and a method for using a host cell to produce a protein or peptide encoded by the polynucleotide comprising culturing the host cell under conditions for expression of the protein or peptide and recovering the protein or peptide from cell culture. The invention also provides purified proteins or peptides encoded by polynucleotides of the invention. The invention further provides a method for using the protein or peptide to screen a plurality of molecules to identify at least one ligand which specifically binds the protein. In one embodiment, the molecules to be screened are selected from agonists, antagonists, antibodies, DNA molecules, peptides, peptide nucleic acids, proteins, and RNA molecules,.

The invention provides a method of using a protein or peptide to identify an antibody which specifically binds the protein or peptide, the method comprising contacting a plurality of antibodies with the protein or peptide under conditions for formation of an antibody:protein/peptide complex, and dissociating the antibody from the antibody:protein/peptide complex, thereby obtaining antibody which specifically binds the protein or peptide. In one aspect, the plurality of antibodies are selected from polyclonal antibodies, monoclonal antibodies, chimeric antibodies, recombinant antibodies, humanized antibodies, single chain antibodies, Fab fragments, F(ab′)₂ fragments, Fv fragments and antibody-peptide fusion proteins. The invention also provides methods for preparing and purifying antibodies. The method for preparing a polyclonal antibody comprises immunizing a animal with protein or peptide under conditions to elicit an antibody response, isolating animal antibodies, attaching the protein or peptide to a substrate, contacting the substrate with isolated antibodies under conditions to allow specific binding to the protein or peptide, dissociating the antibodies from the protein or peptide, thereby obtaining purified polyclonal antibodies. The method for preparing a monoclonal antibodies comprises immunizing a animal with a protein or peptide under conditions to elicit an antibody response, isolating antibody producing cells from the animal, fusing the antibody producing cells with immortalized cells in culture to form monoclonal antibody producing hybridoma cells, culturing the hybridoma cells, and isolating monoclonal antibodies from culture.

The invention provides purified antibodies which specifically bind a protein or peptide. The invention also provides a method for using an antibody to detect expression of a protein in a sample, the method comprising combining the antibody with a sample under conditions for formation of antibody:protein complexes; and detecting complex formation, wherein complex formation indicates expression of the protein in the sample. In one aspect, the antibody is attached to a substrate. In another aspect, the amount of complex formation when compared to standards is diagnostic of ovarian cancer. The invention further provides a method for immunopurification of a protein comprising attaching an antibody to a substrate, exposing the antibody to a sample containing protein under conditions to allow antibody:protein complexes to form, dissociating the protein from the complex, and collecting purified protein.

The invention provides a composition comprising a polynucleotide, a protein, or an antibody that specifically binds a protein or peptide for use in detecting or treating ovarian cancer.

### BRIEF DESCRIPTION OF THE SEQUENCE LISTING

The Sequence Listing provides polynucleotides comprising the nucleic acid sequences of SEQ ID NOs: 1-9. Each sequence is identified by a sequence identification number (SEQ ID NO) and by the Incyte number with which the sequence was first identified.

### DESCRIPTION OF THE INVENTION

It must be noted that as used herein and in the appended claims, the singular forms “a”, “an”, and “the” include the plural reference unless the context clearly dictates otherwise. Thus, for example, a reference to “a host cell” includes a plurality of such host cells, and a reference to “an antibody” is a reference to one or more antibodies and equivalents thereof known to those skilled in the art, and so forth.

Definitions

“Antibody” refers to intact immunoglobulin molecule, a polyclonal antibody, a monoclonal antibody, a chimeric antibody, a recombinant antibody, a humanized antibody, single chain antibodies, a Fab fragment, an F(ab′)₂ fragment, an Fv fragment; and an antibody-peptide fusion protein.

“Antigenic determinant” refers to an antigenic or immunogenic epitope, structural feature, or region of an oligopeptide, peptide, or protein which is capable of inducing formation of an antibody which specifically binds the protein. Biological activity is not a prerequisite for immunogenicity.

“Array” refers to an ordered arrangement of at least two polynucleotides, proteins, or antibodies on a substrate. At least one of the polynucleotides, proteins, or antibodies represents a control or standard, and the other polynucleotide, protein, or antibody of diagnostic or therapeutic interest. The arrangement of at least two and up to about 40,000 polynucleotides, proteins, or antibodies on the substrate assures that the size and signal intensity of each labeled complex, formed between each polynucleotide and at least one nucleic acid, each protein and at least one ligand or antibody, or each antibody and at least one protein to which the antibody specifically binds, is individually distinguishable.

The “complement” of a polynucleotide of the Sequence Listing refers to a nucleic acid molecule which is completely complementary over its full length and which will hybridize to a complementary nucleic acid molecule under conditions of high stringency.

A “composition” refers to the polynucleotide and a labeling moiety; a purified protein and a pharmaceutical carrier or a heterologous, labeling or purification moiety; an antibody and a labeling moiety or pharmaceutical agent; and the like.

“Differential expression” refers to an increased or unregulated or a decreased or down regulated expression as detected by absence, presence, or at least two-fold change in the amount of transcribed messenger RNA or translated protein in a sample.

An “expression profile” is a representation of gene expression in a sample. A nucleic acid expression profile is produced using sequencing, hybridization, or amplification technologies and mRNAs or cDNAs from a sample. A protein expression profile, although time delayed, mirrors the nucleic acid expression profile and uses labeling moieties or antibodies to detect expression in a sample. The nucleic acids, proteins, or antibodies may be used in solution or attached to a substrate, and their detection is based on methods well known in the art.

A “hybridization complex” is formed between a polynucleotide and a nucleic acid of a sample when the purine of one molecule hydrogen bond with the pyrimidine of the complementary molecule, e.g., 5′-A-G-T-C-3′ base pairs with 3′-T-C-A-G-5′. Hybridization conditions, degree of complementarity and the use of nucleotide analogs affect the efficiency and stringency of hybridization reactions.

“Identity” as applied to nucleic and amino acid sequences, refers to the quantification (usually percentage) of nucleotide or residue matches between at least two sequences aligned using a standardized algorithm such as Smith-Waterman alignment (Smith and Waterman (1981) J Mol Biol 147:195-197), CLUSTALW (Thompson et al. (1994) Nucleic Acids Res 22:4673-4680), or BLAST2 (Altschul et al. (1997) Nucleic Acids Res 25:3389-3402. BLAST2 may be used in a standardized and reproducible way to insert gaps in one of the sequences in order to optimize alignment and to achieve a more meaningful comparison between them. “Similarity” uses the same algorithms but takes conservative substitution of nucleotides and residues into account. In proteins, similarity exceeds identity in that substitution of a valine for a leucine or isoleucine, is counted in calculating the reported percentage. Substitutions which are considered to be conservative are well known in the art.

“Isolated or “purified” refers to any molecule or compound that is separated from its natural environment and is from about 60% free to about 90% free from other components with which it is naturally associated.

“Labeling moiety” refers to any reporter molecule including radionuclides, enzymes, fluorescent, chemiluminescent, or chromogenic agents, substrates, cofactors, inhibitors, or magnetic particles than can be attached to or incorporated into a polynucleotide, protein, or antibody. Visible labels include but are not limited to anthocyanins, green fluorescent protein (GFP), βglucuronidase, luciferase, Cy3 and Cy5, and the like. Radioactive markers include radioactive forms of hydrogen, iodine, phosphorous, sulfur, and the like.

“Ligand” refers to any agent, molecule, or compound which will bind specifically to a polynucleotide or to an epitope of a protein. Such ligands stabilize or modulate the activity of polynucleotides or proteins and may be composed of inorganic and/or organic substances including minerals, cofactors, nucleic acids, proteins, carbohydrates, fats, and lipids.

“Markers for ovarian cancer” refers to polynucleotides are useful in the diagnosis, prognosis, or treatment of ovarian cancer. Typically, this means that the marker gene is only expressed or differentially expressed in samples from patients with ovarian cancer.

“Ovarian cancer” includes any tumor of the ovary of primary epithelial origin and specifically refers to serous papillary carcinoma (also known as serous cystadenocarcinoma), mucinous cystadenocarcinoma, endometrioid and mesonephric malignancies, ovarian adenocarcinomas, and borderline ovarian carcinomas.

“Polynucleotide” refers to an isolated cDNA, nucleic acid molecule, or any fragment thereof that contains from about 400 to about 12,000 nucleotides. It may have originated recombinantly or synthetically, may be double-stranded or single-stranded, may represent coding and noncoding 3′ or 5′ sequence, generally lacks introns, and can be combined with vitamins, minerals, carbohydrates, lipids, proteins, or other nucleic acids to perform a particular activity or to form a useful composition.

The phrase “polynucleotide encoding a protein” refers to a nucleic acid whose sequence closely aligns with sequences that encode conserved regions, motifs or domains identified by employing analyses well known in the art. These analyses include BLAST (Basic Local Alignment Search Tool; Altschul (1993) J Mol Evol 36:290-300; Altschul et al. (1990) J Mol Biol 215:403-410) and BLAST2 (Altschul et al. (1997) Nucleic Acids Res 25:3389-3402) which provide identity within the conserved region. Brenner et al. (1998; Proc Natl Acad Sci 95:6073-6078) who analyzed BLAST for its ability to identify structural homologs by sequence identity found 30% identity is a reliable threshold for sequence alignments of at least 150 residues and 40% is a reasonable threshold for alignments of at least 70 residues (Brenner, page 6076, column 2).

“Probe” refers to a cDNA that hybridizes to at least one nucleic acid in a sample. Where targets are single-stranded, probes are complementary single strands. Probes can be labeled with reporter molecules for use in hybridization reactions including Southern, northern, in situ, dot blot, array, and like technologies or in screening assays.

“Protein” refers to a polypeptide or any portion thereof. A “portion” of a protein refers to that length of amino acid sequence which would retain at least one biological activity, a domain identified by PFAM (Washington University, St Louis, Mo.) or PRINTS analysis or an antigenic determinant of the protein identified using Kyte-Doolittle algorithms of the PROTEAN program (DNASTAR, Madison, Wis.).

“Sample” is used in its broadest sense as containing nucleic acids, proteins, and antibodies. A sample may comprise a bodily fluid such as ascites, blood, lymph, semen, sputum, urine and the like; the soluble fraction of a cell preparation, or an aliquot of media in which cells were grown; a chromosome, an organelle, or membrane isolated or extracted from a cell; genomic DNA, RNA, or cDNA in solution or bound to a substrate; a cell; a tissue, a tissue biopsy, or a tissue print; buccal cells, skin, a hair or hair follicle; and the like.

“Specific binding” refers to a special and precise interaction between two molecules which is dependent upon their structure, particularly their molecular side groups. For example, the intercalation of a regulatory protein into the major groove of a DNA molecule or the binding between an epitope of a protein and an agonist, antagonist, or antibody.

“Substrate” refers to any rigid or semi-rigid support to which cDNAs, proteins, or antibodies are bound and includes membranes, filters, chips, slides, wafers, fibers, magnetic or nonmagnetic beads, gels, capillaries or other tubing, plates, polymers, and microparticles with a variety of surface forms including wells, trenches, pins, channels and pores.

A “transcript image” (TI) is an expression profile of gene activity in a particular tissue at a particular time. TI provides assessment of the relative abundance of expressed polynucleotides in the cDNA libraries of an EST database as described in U.S. Pat. No. 5,840,484, incorporated herein by reference.

“Variant” refers to molecules that are recognized variations of a protein or the polynucleotides that encodes it. Splice variants may be determined by BLAST score, wherein the score is at least 100, and most preferably at least 400. Allelic variants have a high percent identity to the cDNAs and may differ by about three bases per hundred bases. “Single nucleotide polymorphism” (SNP) refers to a change in a single base as a result of a substitution, insertion or deletion. The change may be conservative (purine for purine) or non-conservative (purine to pyrimidine) and may or may not result in a change in an encoded amino acid or its secondary, tertiary, or quaternary structure.

The Invention

The present invention identifies a set of polynucleotides, SEQ ID NOs: 1-9 and the complements thereof, that serve as diagnostic markers for ovarian cancer, particularly serous papillary carcinoma (CA). In particular, the method described below identifies polynucleotides cloned from mRNA transcripts which are present or differentially expressed in ovarian cancer. These polynucleotides and the proteins or peptides which they encode and antibodies which specifically bind the proteins or peptides are useful in diagnosis, prognosis, treatment, and evaluation of therapies for ovarian cancer.

The method disclosed below provides for the identification of polynucleotides that are expressed in a plurality of libraries. The polynucleotides originate from human cDNA libraries derived from a variety of sources. These polynucleotides can also be selected from a variety of sequence types including, but not limited to, expressed sequence tags (ESTs), assembled polynucleotides, full length coding regions, promoters, introns, enhancers, 5′ untranslated regions, and 3′ untranslated regions. To have statistically significant analytical results, the polynucleotides are expressed in at least five cDNA libraries.

The cDNA libraries used in the analysis can be obtained from any human tissue including but not limited to adrenal gland, biliary tract, bladder, blood cells, blood vessels, bone marrow, brain, bronchus, cartilage, chromaffin system, colon, connective tissue, cultured cells, embryonic stem cells, endocrine glands, epithelium, esophagus, fetus, ganglia, heart, hypothalamus, immune system, intestine, islets of Langerhans, kidney, larynx, liver, lung, lymph, muscles, neurons, ovary, pancreas, penis, peripheral nervous system, phagocytes, pituitary, placenta, pleura, prostate, salivary glands, seminal vesicles, skeleton, spleen, stomach, testis, thymus, tongue, ureter, and uterus.

The polynucleotides claimed herein were highly specific to ovary and represent those sequences most highly associated with ovarian cancers. The number of cDNA libraries selected can range from as few as 5 to greater than 10,000 and preferably, the number of the cDNA libraries is greater than 500.

In this analysis, 1222 of the 1292 human cDNA libraries containing 40,285 gene bins were used. The libraries contain tissues from surgical samples, biopsies, and cell lines; 41 of these libraries were made from ovary cells and tissues.

In a preferred embodiment, the claimed polynucleotides are assembled from related sequences, such as sequence fragments derived from a single transcript. Assembly of the polynucleotide can be performed using sequences of various types including, but not limited to, ESTs, extensions of the ESTs, shotgun sequences from a cloned insert, or full length cDNAs. In a most preferred embodiment, the polynucleotides are derived from human sequences that have been assembled using the algorithm disclosed in U.S. Pat. No. 9,276,534, filed Mar. 25, 1999, incorporated herein by reference.

Experimentally, differential expression of the polynucleotides can be evaluated by methods including, but not limited to, differential display by spatial immobilization or by gel electrophoresis, genome mismatch scanning, representational difference analysis, microarray analysis and transcript imaging. Any of these methods can be used alone or in combination; in the present case, the preferred method is presented below.

The Method

The method for identifying polynucleotides that exhibit a statistically significant expression pattern in ovary, specifically in ovarian cancer, and particularly in serous papillary carcinoma, is presented below. First, the presence or absence of a polynucleotide in a cDNA library is defined: a polynucleotide is present when at least one cDNA fragment corresponding to that polynucleotide is detected in a cDNA sample taken from the library, and a polynucleotide is absent when no corresponding cDNA fragment is detected in the sample. This method was used with the data in the LIFESEQ GOLD database (Incyte Genomics, Palo Alto, Calif.).

To determine whether a polynucleotide, G, is ovary specific, two statistical tests are applied. In the first test, the significance of gene expression is evaluated using a probability method to measure a due-to-chance probability of the expression. Two dichotomous variables are used to classify the 1222 cDNA libraries, X which determines whether G is present (P) or absent (A), and Y which determines whether the cDNA library is from ovary (O) or not (Θ). Occurrence data in the various categories is summarized in the following contingency table.

| | Ovary | Non-ovary |
|---|---|---|
| G present | PO | P⊖ |
| G absent | AO | A⊖ |

If polynucleotide G is ovary-specific, a positive association between the two variables X and Y is expected; that is, a significant number of libraries should fall into the PO and AΘ categories. To evaluate the significance in statistical terms, the following question is asked: if the null hypothesis were true—that is, the presence of polynucleotide G were completely independent of whether the tissue is ovary or not—how likely is it that the result occurred by chance. This is provided by applying the Fisher Exact probability test and examining the p-value (Agresti (1990) Categorical Data Analysis, John Wiley & Sons, New York, N.Y.; Rice (1988) Mathematical Statistics and Data Analysis, Duxbury Press, Pacific Grove, Calif.). The smaller the p-value, the less likely that the association between X and Y is due-to-chance.

To illustrate, if a polynucleotide was detected in eight of the 1222 cDNA libraries and six of those were from ovary, the corresponding contingency table would be:

| | Ovary | Non-ovary |
|---|---|---|
| G present | 6 | 2 |
| G absent | 40 | 1174 |

and the Fisher Exact p-value would be 5.4⁻⁰⁸, which indicates that the polynucleotide is ovary specific.

In the second test, the EST counts of polynucleotide G from all libraries that were taken from the same tissue are combined and the sum is used as a measure of the expression level in that tissue. In particular, the combined EST count of G in ovary libraries (N_{GO}) is compared to the total number of ESTs for all polynucleotides in ovary libraries (N_{O}) to derive an estimate of the relative abundance of G transcripts in ovary. Similarly, the combined EST count of G in non-ovary libraries (N_{GO}) is compared with the total number of ESTs in non-ovary libraries (N_{Θ}). These values are used to define a likelihood score

L=log2 (N_{GO}/N_{O})/(N_{GΘ}/N_{Θ}),

which reflects how many times more likely it is for the transcript of polynucleotide G to be found in ovary versus non-ovary tissue. For the polynucleotide shown in the contingency table above, the respective counts are N_{GO}=11, N_{O}=108756, N_{GΘ}=3, and N_{Θ}=3556776, which give rise to L=log2(120)=6.91. Because the likelihood score is susceptible to the counting errors that exist in some libraries, the likelihood score is only used as a secondary measure.

In other words, polynucleotides with a significant Fisher Exact p-value of P<1e⁻⁵, are only considered to be ovary-specific if L>5.5. This two-step filtering was found to select most polynucleotides known to function in ovary without including any false positives. Note that the definition of L is flawed when N_{GO}=0 or N_{GΘ}=0. In this case, L>5.5 is considered only when N_{GΘ} and N_{GO}≠0.

Using this method, polynucleotides that exhibit significant association for ovarian cancer have been identified. These polynucleotides, SEQ ID NOs: 1-9 and the complements thereof are useful for the diagnosis, prognosis, and treatment of and evaluation of therapies for ovarian cancer, particularly serous papillary carcinoma. Further, a protein or peptide encoded by any of the polynucleotides can be used as a diagnostic, as a potential therapeutic, as a target for the identification or development of therapeutics, or for producing antibodies which specifically bind the protein or peptide. These antibodies are useful in the diagnosis, prognosis, and treatment of ovarian cancer, particularly serous papillary carcinoma.

In one embodiment, the invention encompasses a combination comprising a plurality of polynucleotides having the nucleic acid sequences of SEQ ID NOs: 1-9 or the complements thereof. These nine polynucleotides are shown by the method of the present invention, specifically in EXAMPLE IV, to have significant, specific expression in ovarian cancer, particularly serous papillary carcinoma. The invention also provides a polynucleotide and its complement, and methods for using a polynucleotide selected from SEQ ID NOs: 1-9.

An expression profile produced using a transcript image is presented in EXAMPLE V. The TI clearly supports the expression of SEQ ID NOs: 1-9 in ovarian cancer, particularly serous papillary carcinoma.

The polynucleotide or the encoded protein or peptide can be used to search against the GenBank primate (pri), rodent (rod), mammalian (mam), vertebrate (vrtp), and eukaryote (eukp) databases, SwissProt, BLOCKS (Bairoch et al. (1997) Nucleic Acids Res 25:217-221), PFAM, and other databases that contain previously identified and annotated motifs, sequences, and gene functions. Methods that search for primary sequence patterns with secondary structure gap penalties (Smith et al. (1992) Protein Engineering 5:35-51) as well as algorithms such as Basic Local Alignment Search Tool (BLAST; Altschul (1993) J Mol Evol 36:290-300; Altschul et al. (1990) J Mol Biol 215:403-410), BLOCKS (Henikoff and Henikoff (1991) Nucleic Acids Res 19:6565-6572), Hidden Markov Models (HMM; Eddy (1996) Cur Opin Str Biol 6:361-365; Sonnhammer et al. (1997) Proteins 28:405-420), and the like, can be used to manipulate and analyze nucleotide and amino acid sequences. These databases, algorithms and other methods are well known in the art and are described in Ausubel et al. (1997; Short Protocols in Molecular Biology, John Wiley & Sons, New York, N.Y., unit 7.7) and in Meyers (1995; Molecular Biology and Biotechnology, Wiley V C H, New York, N.Y., p 856-853).

Also encompassed by the invention are polynucleotides that are capable of hybridizing to SEQ ID NOs: 1-9, under stringent conditions. Stringent conditions can be defined by salt concentration, temperature, and other chemicals and conditions well known in the art (Ausubel (supra) unit 2, pp. 1-41; unit 4, pp. 22-27). Conditions can be selected by varying the concentrations of salt in the prehybridization, hybridization, and wash solutions or by varying the hybridization and wash temperatures. With some substrates, the temperature can be decreased by adding formamide to the prehybridization and hybridization solutions.

Hybridization can be performed at low stringency, with buffers such as 5×SSC (saline sodium citrate) with 1% sodium dodecyl sulfate (SDS) at 60C., which permits complex formation between two nucleic acid sequences that contain some mismatches. Subsequent washes are performed at higher stringency with buffers such as 0.2×SSC with 0.1% SDS at either 45C. (medium stringency) or 68C. (high stringency), to maintain hybridization of only those complexes that contain completely complementary sequences. Background signals can be reduced by the use of detergents such as SDS, sarcosyl, or TRITON X-100 (Sigma-Aldrich, St. Louis, Mo.), and/or a blocking agent, such as salmon sperm DNA. Hybridization methods are described in detail in Ausubel (supra, units 2.8-2.11, 3.18-3.19 and 4-6-4.9) and Sambrook et al. (1989; Molecular Cloning A Laboratory Manual, Cold Spring Harbor Press, Plainview, N.Y.)

A polynucleotide can be extended utilizing a partial nucleotide sequence and employing various methods such as PCR and shotgun cloning which are well known in the art. These methods can be used to extend upstream or downstream to obtain a full length sequence or to recover useful untranslated regions (UTRs), such as promoters and other regulatory elements. For PCR extensions, an XL-PCR kit (Applied Biosystems (ABI), Foster City, Calif.), nested primers, and commercially available cDNA libraries (Invitrogen, Carlsbad, Calif.) or genomic libraries (Clontech, Palo Alto, Calif.) can be used to extend the sequence. For all PCR-based methods, primers can be designed using commercially available software (LASERGENE software, DNASTAR, Madison, Wis.) to be about 15 to 30 nucleotides in length, to have a GC content of about 50%, and to form a hybridization complex at temperatures of about 68C. to 72C.

In another aspect of the invention, the polynucleotide can be cloned into a recombinant vector that directs the expression of the protein, peptide, or structural or functional portions thereof, in host cells. Due to the inherent degeneracy of the genetic code, other DNA sequences which encode substantially the same or a functionally equivalent amino acid sequence can be produced and used to express the protein encoded by the polynucleotide. The nucleotide sequences of the present invention can be engineered using methods generally known in the art in order to alter the nucleotide sequences for a variety of purposes including, but not limited to, modification of the cloning, processing, and/or expression of the gene product. DNA shuffling by random fragmentation and PCR reassembly of gene fragments and synthetic oligonucleotides can be used to engineer the nucleotide sequences. For example, oligonucleotide-mediated site-directed mutagenesis can be used to introduce mutations that create new restriction sites, alter glycosylation patterns, change codon preference, produce splice variants, and so forth.

In order to express a biologically active protein, the polynucleotide or derivatives thereof, can be inserted into an expression vector which contains the elements for transcriptional and translational control of the inserted coding sequence in a particular host. These elements can include regulatory sequences, such as enhancers, constitutive and inducible promoters, and 5′ and 3′ untranslated regions. Methods which are well known to those skilled in the art can be used to construct such expression vectors. These methods include in vitro recombinant DNA techniques, synthetic techniques, and in vivo genetic recombination (Sambrook, supra; Ausubel, supra).

A variety of expression vector/host cell systems can be utilized to express the polynucleotide. These include, but are not limited to, microorganisms such as bacteria transformed with recombinant bacteriophage, plasmid, or cosmid expression vectors; yeast transformed with yeast expression vectors; insect cell systems infected with baculovirus vectors; plant cell systems transformed with viral or bacterial expression vectors; or animal cell systems. For long term production of recombinant proteins in mammalian systems, stable expression in cell lines is preferred. For example, the polynucleotide can be transformed into cell lines using expression vectors which can contain viral origins of replication and/or endogenous expression elements and a selectable or visible marker gene on the same or on a separate vector. The invention is not to be limited by the vector or host cell employed.

In general, host cells that contain the polynucleotide and that express the protein can be identified by a variety of procedures known to those of skill in the art. These procedures include, but are not limited to, DNA-DNA or DNA-RNA hybridizations, PCR amplification, and protein bioassay or immunoassay techniques which include membrane, solution, or chip based technologies for the detection and/or quantification of nucleic acid or amino acid sequences. Immunological methods for detecting and measuring the expression of the protein using either specific polyclonal or monoclonal antibodies are known in the art. Examples of such techniques include enzyme-linked immunosorbent assays (ELISAs), radioimmunoassays (RIAs), and fluorescence activated cell sorting (FACS).

Host cells transformed with the polynucleotide can be cultured under conditions for the expression and recovery of the protein from cell culture. The protein produced by a transgenic cell can be secreted or retained intracellularly depending on the sequence and/or the vector used. As will be understood by those of skill in the art, expression vectors containing the polynucleotide can be designed to contain signal sequences which direct secretion of the protein through a prokaryotic or eukaryotic cell membrane.

In addition, a host cell strain can be chosen for its ability to modulate expression of the inserted sequences or to process the expressed protein in the desired fashion. Such modifications of the protein include, but are not limited to, acetylation, carboxylation, glycosylation, phosphorylation, lipidation, and acylation. Post-translational processing which cleaves a “prepro” form of the protein can also be used to specify protein targeting, folding, and/or activity. Different host cells which have specific cellular machinery and characteristic mechanisms for post-translational activities (e.g., CHO, HeLa, MDCK, HEK293, and WI38) are available from the ATCC (Manassas, Va.) and can be chosen to ensure the correct modification and processing of the expressed protein.

In another embodiment of the invention, natural, modified, or recombinant nucleic acid sequences are ligated to a heterologous sequence resulting in translation of a fusion protein containing heterologous protein moieties in any of the aforementioned host systems. Such heterologous protein moieties facilitate purification of fusion proteins using commercially available affinity matrices. Such moieties include, but are not limited to, glutathione S-transferase, maltose binding protein, thioredoxin, calmodulin binding peptide, 6-His, FLAG, c-myc, hemaglutinin, and monoclonal antibody epitopes.

In another embodiment, the polynucleotides, wholly or in part, are synthesized using chemical or enzymatic methods well known in the art (Caruthers et al. (1980) Nucl Acids Symp Ser (7) 215-233; Ausubel, supra). For example, peptide synthesis can be performed using various solid-phase techniques (Roberge et al. (1995) Science 269:202-204), and machines such as the 431A peptide synthesizer (ABI) can be used to automate synthesis. If desired, the amino acid sequence can be altered during synthesis and/or combined with sequences from other proteins to produce a variant.

Screening, Diagnostics and Therapeutics

The polynucleotides are particularly useful as markers in diagnosis, prognosis, treatment, and selection and evaluation of therapies for ovarian cancer. The polynucleotides can also be used to screen a plurality of molecules for specific binding affinity. The assay can be used to screen a plurality of DNA molecules, RNA molecules, peptide nucleic acids, peptides, ribozymes, antibodies, agonists, antagonists, immunoglobulins, inhibitors, proteins including transcription factors, enhancers, repressors, and drugs and the like which regulate the activity of the polynucleotide in the biological system. An exemplary assay involves providing a plurality of molecules, combining the polynucleotide or a composition thereof with the plurality of molecules under conditions to allow specific binding, and detecting specific binding to identify at least one molecule which specifically binds the polynucleotide.

Similarly proteins or peptides can be used to screen libraries of molecules or compounds in any of a variety of screening assays. The protein or peptide employed in such screening can be free in solution, affixed to an abiotic or biotic substrate (e.g., borne on a cell surface), or located intracellularly. Specific binding between the protein and the molecule can be measured. The assay can be used to screen a plurality of DNA molecules, RNA molecules, PNAs, peptides, mimetics, ribozymes, antibodies, agonists, antagonists, immunoglobulins, inhibitors, peptides, polypeptides, drugs and the like, which specifically bind the protein. One method for high throughput screening using very small assay volumes and very small amounts of test compound is described in Burbaum et al. U.S. Pat. No. 5,876,946, incorporated herein by reference, which screens large numbers of molecules for enzyme inhibition or receptor binding.

In one preferred embodiment, the polynucleotides are used for diagnostic purposes to determine the absence, presence, or differential—increased or decreased compared to a normal or standard—expression of the gene. The polynucleotide consists of complementary RNA and DNA molecules, branched nucleic acids, and/or PNAs. In one alternative, the polynucleotides are used to detect and quantify gene expression in samples in which expression of the polynucleotide is indicative of ovarian cancer. In another alternative, the polynucleotide can be used to detect genetic polymorphisms associated with ovarian cancer. These polymorphisms can be detected in transcripts or genomic sequences.

The specificity of the probe is determined by whether it is made from a unique region, a regulatory region, or from a conserved motif. Both probe specificity and the stringency of hybridization or amplification (maximal, high, intermediate, or low) will determine whether the probe identifies only naturally occurring, exactly complementary sequences, allelic variants, or related sequences. Probes designed to detect related sequences should have at least 50% sequence identity and to detect a sequence having a polymorphism preferably 94% sequence identity.

Methods for producing hybridization probes include the cloning of the polynucleotide into vectors for the production of RNA probes. Such vectors are known in the art, are commercially available, and can be used to synthesize RNA probes in vitro by adding RNA polymerases and labeled nucleotides. Hybridization probes can incorporate nucleotides labeled by a variety of reporter groups including, but not limited to, radionuclides such as ³²P or ³⁵S, enzymatic labels such as alkaline phosphatase coupled to the probe via avidin/biotin coupling systems, fluorescent labels, and the like. The labeled polynucleotides can be used in Southern or northern analysis, dot or slot blot, or other membrane-based technologies; in PCR technologies; and in microarrays utilizing samples from subjects to detect differential expression.

The polynucleotide can be labeled by standard methods and added to a sample from a subject under conditions for the formation and detection of hybridization complexes. After incubation the sample is washed, and the signal associated with hybrid complex formation is quantitated and compared with a standard value. Standard values are derived from any control sample, typically one that is free of the suspect disease. If the amount of signal in the subject sample is altered in comparison to the standard value, then the presence of differential expression in the sample indicates the presence of the disease. Qualitative and quantitative methods for comparing the hybridization complexes formed in subject samples with previously established standards are well known in the art.

Such assays can also be used to evaluate the efficacy of a particular therapeutic treatment regimen in animal studies, in clinical trials, or to monitor the treatment of an individual subject. Once the presence of disease is established and a treatment protocol is initiated, hybridization or amplification assays can be repeated on a regular basis to determine if the level of expression in the patient begins to approximate that which is observed in a healthy subject. The results obtained from successive assays can be used to show the efficacy of treatment over a period ranging from several days to many years.

The polynucleotides can be used as a group or alone for the diagnosis of ovarian cancer. The polynucleotides can also be used on a substrate such as microarray to monitor the expression patterns. The microarray can also be used to identify splice variants, mutations, and polymorphisms. Information derived from analyses of the expression patterns can be used to determine gene function, to understand the genetic basis of a disease, to diagnose a disease, and to develop and monitor the activities of therapeutic agents used to treat a disease. Microarrays can also be used to detect genetic diversity, single nucleotide polymorphisms which can characterize a particular population, at the genome level.

In yet another alternative, polynucleotides can be used to generate hybridization probes useful in mapping the naturally occurring genomic sequence. Fluorescent in situ hybridization (FISH) can be correlated with other physical chromosome mapping techniques and genetic map data as described in Heinz-Ulrich et al. (In: Meyers (supra) pp. 965-968).

In another embodiment, antibodies or Fabs comprising an antigen binding site that specifically binds the protein can be used for the diagnosis of diseases characterized by the over-or-under expression of the protein. A variety of protocols for measuring protein expression, including ELISAs, RIAs, and FACS, are well known in the art and provide a basis for diagnosing differential, altered or abnormal levels of expression. Standard values for protein expression are established by combining samples taken from healthy subjects, preferably human, with antibody to the protein under conditions for complex formation. The amount of complex formation can be quantitated by various methods, preferably by photometric means. Quantities of the protein expressed in disease samples are compared with standard values. Deviation between standard and subject values establishes the parameters for diagnosing or monitoring disease. Alternatively, one can use competitive drug screening assays in which neutralizing antibodies capable of binding specifically with the protein compete with a test compound. Antibodies can be used to detect the presence of any peptide which shares one or more antigenic determinants with the protein. In one aspect, the antibodies of the present invention can be used for treatment or monitoring therapeutic treatment for ovarian cancer.

In another aspect, the polynucleotide, or its complement, can be used therapeutically for the purpose of expressing mRNA and protein, or conversely to block transcription or translation of the mRNA. Expression vectors can be constructed using elements from retroviruses, adenoviruses, herpes or vaccinia viruses, or bacterial plasmids, and the like. These vectors can be used for delivery of nucleotide sequences to a particular target organ, tissue, or cell population. Methods well known to those skilled in the art can be used to construct vectors to express nucleic acid sequences or their complements (see, e.g., Maulik et al. (1997) Molecular Biotechnology, Therapeutic Applications and Strategies, Wiley-Liss, New York, N.Y.). Alternatively, the polynucleotide or its complement, can be used for somatic cell or stem cell gene therapy. Vectors can be introduced in vivo, in vitro, and ex vivo. For ex vivo therapy, vectors are introduced into stem cells taken from the subject, and the resulting transgenic cells are clonally propagated for autologous transplant back into that same subject. Delivery of the polynucleotide by transfection, liposome injections, or polycationic amino polymers can be achieved using methods which are well known in the art (See, e.g., Goldman et al. (1997) Nature Biotechnol 15:462-466). Additionally, endogenous gene expression can be inactivated using homologous recombination methods which insert an inactive gene sequence into the coding region or other targeted region of the polynucleotide (see, e.g., Thomas et al. (1987) Cell 51:503-512).

Vectors containing the polynucleotide can be transformed into a cell or tissue to express a missing protein or to replace a nonfunctional protein. Similarly a vector constructed to express the complement of the polynucleotide can be transformed into a cell to downregulate the protein expression. Complementary or antisense sequences can consist of an oligonucleotide derived from the transcription initiation site; nucleotides between about positions −10 and +10 from the ATG are preferred. Similarly, inhibition can be achieved using triple helix base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature (see, e.g., Gee et al. In: Huber and Carr (1994) Molecular and Immunologic Approaches, Futura Publishing, Mt. Kisco, N.Y., pp. 163-177).

Ribozymes, enzymatic RNA molecules, can also be used to catalyze the cleavage of mRNA and decrease the levels of particular Minas, such as those comprising the polynucleotides of the invention (see, e.g., Rossi (1994) Current Biology 4: 469-47). Ribozymes can cleave mRNA at specific cleavage sites. Alternatively, ribozymes can cleave mRNAs at locations dictated by flanking regions that form complementary base pairs with the target mRNA. The construction and production of ribozymes is well known in the art and is described in Meyers (supra).

RNA molecules can be modified to increase intracellular stability and half-life. Possible modifications include, but are not limited to, the addition of flanking sequences at the 5′ and/or 3′ ends of the molecule, or the use of phosphorothioate or 2′ O-methyl rather than phosphodiester linkages within the backbone of the molecule. Alternatively, nontraditional bases such as inosine, queosine, and wybutosine, as well as acetyl-, methyl-, thio-, and similarly modified forms of adenine, cytidine, guanine, thymine, and uridine which are not as easily recognized by endogenous endonucleases, can be included.

Further, an antagonist, or an antibody that binds specifically to the protein can be administered to a subject to treat ovarian cancer. The antagonist, antibody, or fragment can be used directly to inhibit the activity of the protein or indirectly to deliver a therapeutic agent to cells or tissues which express the protein. The therapeutic agent can be a cytotoxic agent selected from a group including, but not limited to, abrin, ricin, doxorubicin, daunorubicin, taxol, ethidium bromide, mitomycin, etoposide, tenoposide, vincristine, vinblastine, colchicine, dihydroxy anthracin dione, actinomycin D, diphteria toxin, Pseudomonas exotoxin A and 40, radioisotopes, and glucocorticoid.

Antibodies to the protein can be generated using methods that are well known in the art. Such antibodies can include, but are not limited to, polyclonal, monoclonal, chimeric, and single chain antibodies, Fab fragments, and fragments produced by a Fab expression library. Neutralizing antibodies, such as those which inhibit dimer formation, are especially preferred for therapeutic use. Monoclonal antibodies to the protein can be prepared using any technique which provides for the production of antibody molecules by continuous cell lines in culture. These include, but are not limited to, the hybridoma, the human B-cell hybridoma, and the EBV-hybridoma techniques. In addition, techniques developed for the production of chimeric antibodies can be used (see, e.g., Pound (1998) Immunochemical Protocols, Methods Mol Biol Vol. 80). Alternatively, techniques described for the production of single chain antibodies can be employed. Fabs which contain specific binding sites for the protein can also be generated. Various immunoassays can be used to identify antibodies having the desired specificity. Numerous protocols for competitive binding or immunoradiometric assays using either polyclonal or monoclonal antibodies with established specificities are well known in the art.

Yet further, an agonist of the protein can be administered to a subject to treat or prevent a disease associated with decreased expression, longevity or activity of the protein.

Pharmaceutical Compositions

Pharmaceutical compositions may be formulated and administered, to a subject in need of such treatment, to attain a therapeutic effect. Such compositions contain the instant protein, agonists, antibodies specifically binding the protein, antagonists, inhibitors, or mimetics of the protein. Compositions may be manufactured by conventional means such as mixing, dissolving, granulating, dragee-making, levigating, emulsifying, encapsulating, entrapping, or lyophilizing. The composition may be provided as a salt, formed with acids such as hydrochloric, sulfuric, acetic, lactic, tartaric, malic, and succinic, or as a lyophilized powder which may be combined with a sterile buffer such as saline, dextrose, or water. These compositions may include auxiliaries or excipients which facilitate processing of the active compounds.

Auxiliaries and excipients may include coatings, fillers or binders including sugars such as lactose, sucrose, mannitol, glycerol, or sorbitol; starches from corn, wheat, rice, or potato; proteins such as albumin, gelatin and collagen; cellulose in the form of hydroxypropylmethyl-cellulose, methyl cellulose, or sodium carboxymethylcellulose; gums including arabic and tragacanth; lubricants such as magnesium stearate or talc; disintegrating or solubilizing agents such as the, agar, alginic acid, sodium alginate or cross-linked polyvinyl pyrrolidone; stabilizers such as carbopol gel, polyethylene glycol, or titanium dioxide; and dyestuffs or pigments added for identify the product or to characterize the quantity of active compound or dosage.

These compositions may be administered by any number of routes including oral, intravenous, intramuscular, intra-arterial, intramedullary, intrathecal, intraventricular, transdermal, subcutaneous, intraperitoneal, intranasal, enteral, topical, sublingual, or rectal.

The route of administration and dosage will determine formulation; for example, oral administration may be accomplished using tablets, pills, dragees, capsules, liquids, gels, syrups, slurries, or suspensions; parenteral administration may be formulated in aqueous, physiologically compatible buffers such as Hanks' solution, Ringer's solution, or physiologically buffered saline. Suspensions for injection may be aqueous, containing viscous additives such as sodium carboxymethyl cellulose or dextran to increase the viscosity, or oily, containing lipophilic solvents such as sesame oil or synthetic fatty acid esters such as ethyl oleate or triglycerides, or liposomes. Penetrants well known in the art are used for topical or nasal administration.

Toxicity and Therapeutic Efficacy

A therapeutically effective dose refers to the amount of active ingredient which ameliorates symptoms or condition. For any compound, a therapeutically effective dose can be estimated from cell culture assays using normal and neoplastic cells or in animal models. Therapeutic efficacy, toxicity, concentration range, and route of administration may be determined by standard pharmaceutical procedures using experimental animals.

The therapeutic index is the dose ratio between therapeutic and toxic effects—LD50 (the dose lethal to 50% of the population)/ED50 (the dose therapeutically effective in 50% of the population)—and large therapeutic indices are preferred. Dosage is within a range of circulating concentrations, includes an ED50 with little or no toxicity, and varies depending upon the composition, method of delivery, sensitivity of the patient, and route of administration. Exact dosage will be determined by the practitioner in light of factors related to the subject in need of the treatment.

Dosage and administration are adjusted to provide active moiety that maintains therapeutic effect. Factors for adjustment include the severity of the disease state, general health of the subject, age, weight, and gender of the subject, diet, time and frequency of administration, drug combination(s), reaction sensitivities, and tolerance/response to therapy. Long-acting phannaccutical compositions may be administered every 3 to 4 days, every week, or once every two weeks depending on half-life and clearance rate of the particular composition.

Normal dosage amounts may vary from 0.1 μg, up to a total dose of about 1 g, depending upon the route of administration. The dosage of a particular composition may be lower when administered to a patient in combination with other agents, drugs, or hormones. Guidance as to particular dosages and methods of delivery is provided in the pharmaceutical literature and generally available to practitioners. Further details on techniques for formulation and administration may be found in the latest edition of Remington's Pharmaceutical Sciences (Mack Publishing, Easton, Pa.).

Stem Cells and Their Use

SEQ ID NOs: 1-9 may be useful in the differentiation of stem cells. Eukaryotic stem cells are able to differentiate into the multiple cell types of various tissues and organs and to play roles in embryogenesis and adult tissue regeneration (Gearhart (1998) Science 282:1061-1062; Watt and Hogan (2000) Science 287:1427-1430). Depending on their source and developmental stage, stem cells can be totipotent with the potential to create every cell type in an organism and to generate a new organism, pluripotent with the potential to give rise to most cell types and tissues, but not a whole organism; or multipotent cells with the potential to differentiate into a limited number of cell types. Stem cells can be transfected with polynucleotides which can be transiently expressed or can be integrated within the cell as transgenes.

Embryonic stem (ES) cell lines are derived from the inner cell masses of human blastocysts and are pluripotent (Thomson et al. (1998) Science 282:1145-1147). They have normal karyotypes and express high levels of telomerase which prevents senescence and allows the cells to replicate indefinitely. ES cells produce derivatives that give rise to embryonic epidermal, mesodermal and endodermal cells. Embryonic germ (EG) cell lines, which are produced from primordial germ cells isolated from gonadal ridges and mesenteries, also show stem cell behavior (Shamblott et al. (1998) Proc Natl Acad Sci 95:13726-13731). EG cells have normal karyotypes and appear to be pluripotent.

Organ-specific adult stem cells differentiate into the cell types of the tissues from which they were isolated. They maintain their original tissues by replacing cells destroyed from disease or injury. Adult stem cells are multipotent and under proper stimulation can be used to generate cell types of various other tissues (Vogel (2000) Science 287:1418-1419). Hematopoietic stem cells from bone marrow provide not only blood and immune cells, but can also be induced to transdifferentiate to form brain, liver, heart, skeletal muscle and smooth muscle cells. Similarly mesenchymal stem cells can be used to produce bone marrow, cartilage, muscle cells, and some neuron-like cells, and stem cells from muscle have the ability to differentiate into muscle and blood cells (Jackson et al. (1999) Proc Natl Acad Sci 96:14482-14486). Neural stem cells, which produce neurons and glia, can also be induced to differentiate into heart, muscle, liver, intestine, and blood cells (Kuhn and Svendsen (1999) BioEssays 21:625-630); Clarke et al. (2000) Science 288:1660-1663; Gage (2000) Science 287:1433-1438; and Galli et al. (2000) Nature Neurosci 3:986-991).

Neural stem cells can be used to treat neurological disorders such as Alzheimer disease, Parkinson disease, and multiple sclerosis and to repair tissue damaged by strokes and spinal cord injuries. Hematopoietic stem cells can be used to restore immune function in immunodeficient patients or to treat autoimmune disorders by replacing autoreactive immune cells with normal cells to treat diseases such as multiple sclerosis, scleroderma, rheumatoid arthritis, and systemic lupus erythematosus. Mesenchymal stem cells can be used to repair tendons or to regenerate cartilage to treat arthritis. Liver stem cells can be used to repair liver damage. Pancreatic stem cells can be used to replace islet cells to treat diabetes. Muscle stem cells can be used to regenerate muscle to treat muscular dystrophies (Fontes and Thomson (1999) B M J 319:1-3; Weissman (2000) Science 287:1442-1446; Marshall (2000) Science 287:1419-1421; Marmont (2000) Ann Rev Med 51:115-134).

### EXAMPLES

It is to be understood that this invention is not limited to the particular devices, machines, materials and methods described. Although particular embodiments known at the time the invention was made are described, equivalent embodiments can be used to practice the invention. The described embodiments are provided to illustrate the invention and are not intended to limit the scope of the invention which is limited only by the appended claims.

I cDNA Library Construction

The OVARTUM02 library was constructed at Stratagene (La Jolla, Calif.) from ovarian serous papillary carcinoma tumor tissue removed from a 64-year-old female (STR937219). The tissue was flash frozen, ground in a mortar and pestle, and lysed in a buffer containing guanidinium isothiocyanate. The lysate was extracted twice with a mixture of phenol and chloroform, pH 8.0, and centrifuged over a CsCl cushion. The RNA was precipitated with 0.3 M sodium acetate and 2.5 volumes of ethanol, resuspended in water, and DNAse treated for 15 min at 37C. The polyadenylated RNA was isolated with the OLIGOTEX kit (Qiagen, Chatsworth, Calif.) and used to construct the cDNA library.

The OVARTUP08 cDNA library sequence was obtained from the Cancer Genome Anatomy Project (CGAP: PD Name NCl_CGAP_Ov8). The library was described as being constructed from mRNA made from invasive serous papillary adenocarcinoma removed from an adult female. cDNA was made using an oligo d(T) primer. Double-stranded cDNA was size-selected (average insert size was 600 bp) on an agarose gel and nondirectionally cloned into the pAMP10 vector (Krizman et al. (1996) Cancer Research 56:5380-5383).

II Isolation and Sequencing of cDNAs

First strand cDNA synthesis was accomplished using an oligo d(T) primer/linker which also contained an XhoI restriction site. Second strand synthesis was performed using a combination of DNA polymerase I, E. coli ligase and RNAse H, followed by the addition of an EcoRI adaptor to the blunt ended cDNA. The EcoRI adapted, double-stranded cDNA was then digested with XhoI restriction enzyme and fractionated to obtain sequences which exceeded 800 bp in size. The cDNAs were inserted into the Lambda UNIZAP vector system (Stratagene); then the vector which contains the pBLUESCRIPT phagemid (Stratagene) was transformed into E. coli XL1-BLUEMRF host cells (Stratagene).

The phagemids containing the individual cDNA clones were obtained by the in vivo excision process. Enzymes from both pBLUESCRIPT and a cotransformed f1 helper phage nicked the DNA, initiated new DNA synthesis, and created the smaller, single-stranded circular phagemid molecules which contained the cDNA insert. The phagemid DNA was released, purified, and used to reinfect fresh SOLR host cells (Stratagene). Presence of the β-lactamase gene in the phagemid allowed transformed bacteria to grow on medium containing ampicillin.

In the alternative, plasmid DNA was released from the cells and purified using either the MINIPREP kit (Edge Biosystems, Gaithersburg, Md.) or the REAL PREP 96 plasmid kit (Qiagen). A kit consists of a 96-well block with reagents for 960 purifications. The recommended protocol was employed except for the following changes: 1) the bacteria were cultured in 1 ml of sterile TERRIFIC BROTH (BD Biosciences, San Jose, Calif.) with carbenicillin at 25 mg/l and glycerol at 0.4%; 2) after 19 hours incubation, the cells were lysed in 0.3 ml of lysis buffer; and 3) following isopropanol precipitation, the plasmid DNA pellet was resuspended in 0.1 ml of distilled water. After the last step in the protocol, samples were transferred to a 96-well block for storage at 4C.

The cDNAs were prepared using a MICROLAB 2200 system (Hamilton, Reno, Nev.) in combination with DNA ENGINE thermal cyclers (MJ Research, Watertown, Mass.). The cDNAs were sequenced by the method of Sanger and Coulson (1975; J Mol Biol 94:441-448) using PRISM 377 DNA sequencing systems (ABI). Most of the sequences were sequenced using standard ABI protocols and kits at solution volumes of 0.25×-1.0×. In the alternative, some of the sequences were sequenced using solutions and dyes from Amersham Pharmacia Biotech (APB).

III Assembly of Polynucleotides and Characterization of Sequences

The polynucleotides used for co-expression analysis were derived from cDNA, extension, and shotgun sequences and were assembled and analyzed using a combination of software programs which utilize algorithms well known to those skilled in the art (Meyers, supra, pp 856-853).

The polynucleotides of this application were compared with assembled consensus sequences or templates found in the LIFESEQ GOLD database (Incyte Genomics). Component sequences from polynucleotide, extension, full length, and shotgun sequencing projects were subjected to PHRED analysis and assigned a quality score. All sequences with an acceptable quality score were subjected to various pre-processing and editing pathways to remove low quality 3′ ends, vector and linker sequences, polyA tails, Alu repeats, mitochondrial and ribosomal sequences, and bacterial contamination sequences. Edited sequences had to be at least 50 bp in length, and low-information sequences and repetitive elements such as dinucleotide repeats, Alu repeats, and the like, were replaced by “Ns” or masked.

Edited sequences were subjected to assembly procedures in which the sequences were assigned to gene bins. Each sequence could only belong to one bin, and sequences in each bin were assembled to produce a template. Newly sequenced components were added to existing bins using BLAST and CROSSMATCH. To be added to a bin, the component sequences had to have a BLAST quality score greater than or equal to 150 and an alignment of at least 82% local identity. The sequences in each bin were assembled using PHRAP. Bins with several overlapping component sequences were assembled using DEEP PHRAP. The orientation of each template was determined based on the number and orientation of its component sequences.

Bins were compared to one another and those having local similarity of at least 82% were combined and reassembled. Bins having templates with less than 95% local identity were split. Templates were subjected to analysis by STITCHER/EXON MAPPER algorithms (Incyte Genomics) that analyze the probabilities of the presence of splice variants, alternatively spliced exons, splice junctions, differential expression of alternative spliced genes across tissue types or disease states, and the like. Assembly procedures were repeated periodically, and templates were annotated using BLAST against GenBank databases such as GBpri. An exact match was defined as having from 95% local identity over 200 base pairs through 100% local identity over 100 base pairs and a homolog match as having an E-value (or probability score) of ≦1×10⁻⁸. The templates were also subjected to frameshift FASTx against GENPEPT, and homolog match was defined as having an E-value of ≦1×10⁻⁸. Template analysis and assembly was described in U.S. Ser. No. 09/276,534, filed Mar. 25, 1999.

Following assembly, templates were subjected to BLAST, motif, and other functional analyses and categorized in protein hierarchies using methods described in U.S. Ser. No. 08/812,290 and U.S. Ser. No. 08/811,758, both filed Mar. 6, 1997; in U.S. Ser. No. 08/947,845, filed Oct. 9, 1997; and in U.S. Ser. No. 09/034,807, filed Mar. 4, 1998. Then templates were analyzed by translating each template in all three forward reading frames and searching each translation against the PFAM database of hidden Markov model-based protein families and domains using the HMMER software package (Washington University School of Medicine, St. Louis, Mo.).

The BLAST software suite, freely available sequence comparison algorithms (NCBI, Bethesda, Md.), includes various sequence analysis programs including “blastn” that is used to align nucleic acid molecules and BLAST 2 that is used for direct pairwise comparison of either nucleic or amino acid molecules. BLAST programs are commonly used with gap and other parameters set to default settings, e.g.: Matrix: BLOSUM62; Reward for match: 1; Penalty for mismatch: −2; Open Gap: 5 and Extension Gap: 2 penalties; Gap×drop-off: 50; Expect: 10; Word Size: 11; and Filter: on. Identity or similarity is measured over the entire length of a sequence or some smaller portion thereof. Brenner et al. (1998; Proc Natl Acad Sci 95:6073-6078, incorporated herein by reference) analyzed the BLAST for its ability to identify structural homologs by sequence identity and found 30% identity is a reliable threshold for sequence alignments of at least 150 residues and 40%, for alignments of at least 70 residues.

The polynucleotide and any encoded protein were further queried against public databases such as the GenBank rodent, mammalian, vertebrate, prokaryote, and eukaryote databases, SwissProt, BLOCKS, PRINTS, PFAM, and Prosite.

IV Expression of Polynucleotides in Ovarian Cancer

Using the data in the LIFESEQ GOLD database (Incyte Genomics), nine polynucleotides that showed highly significant expression, a cutoff p-value of less than 0.00001 (P<1e⁻⁵), in ovarian cancer were identified. The statistical method presented in the DESCRIPTION OF THE INVENTION was used to identify these polynucleotides among approximately five million cDNAs assigned to one of the 40,285 gene bins. The algorithms identified polynucleotides expressed with high specificity in ovary, in ovarian cancer and particularly in serous papillary carcinoma. Table 1 shows the expression for each polynucleotide as identified by its SEQ ID NO.

TABLE 1
POLYNUCLEOTIDES HIGHLY AND SPECIFICALLY EXPRESSED
IN OVARY AND OVARIAN CANCER

| | (log 2) | | | # O | # O Libs | # O | | | | | |
|---|---|---|---|---|---|---|---|---|---|---|---|
| | O/⊖ | # O | # ⊖ | Tumor | w/Other | Normal | | | | | |
| SEQ ID | (P) | Libs | Libs | Libs | Diseases | Libs | P O | P ⊖ | A O | A ⊖ | p-value |
| 1 | 6.03 | 10 | 5 | 10 | 0 | 0 | 4 | 3 | 42 | 1173 | 5.70E−05 |
| 2 | 7.03 | 4 | 1 | 3 | 1 | 0 | 3 | 1 | 43 | 1175 | 0.00019 |
| 3 | 7.03 | 8 | 2 | 8 | 0 | 0 | 3 | 2 | 43 | 1174 | 0.00047 |
| 4 | 6.25 | 7 | 3 | 7 | 0 | 0 | 3 | 2 | 43 | 1174 | 0.00047 |
| 5 | 6.73 | 13 | 4 | 13 | 0 | 0 | 5 | 2 | 41 | 1174 | 1.20E−06 |
| 6 | 6.91 | 11 | 3 | 11 | 0 | 0 | 6 | 2 | 40 | 1174 | 5.40E−08 |
| 7 | 6.77 | 10 | 3 | 10 | 0 | 0 | 3 | 3 | 43 | 1173 | 0.00092 |
| 8 | 6.62 | 6 | 2 | 6 | 0 | 0 | 6 | 2 | 40 | 1174 | 5.40E−08 |
| 9 | 6.16 | 35 | 16 | 34 | 0 | 1 | 6 | 15 | 40 | 1161 | 7.20E−05 |
| Legend: Column 1 shows the SEQ ID NO; column 2, the expression ratio (log2) of ovary vs. non-ovary, polynucleotide present; column 3, number of transcripts in ovary libraries; column 4, number of transcripts in non-ovary libraries; column 5, number of transcripts in ovary tumor libraries, column 6, number of transcripts in diseased, non-ovary libraries; column 7, number of transcripts in normal ovary libraries; column 8, number of normal ovary libraries, polynucleotide | | | | | | | | | | | |
| # present; column 9, number of non-ovary libraries polynucleotide present; column 10, number of ovary libraries, polynucleotide absent; column 11, number of non-ovary libraries, polynucleotide absent; and column 12, Fisher Exact p-value for ovary vs. non-ovary. | | | | | | | | | | | |

V Transcript Imaging

The transcript image below was produced by sequencing cDNAs and then naming, matching, and counting all copies of related clones and arranging them in order of abundance. The process of producing a comparative transcript image was fully described in U.S. Pat. No. 5,840,484, incorporated herein by reference.

The general categories for which transcript image data is available include cardiovascular system, connective tissue, digestive system, embryonic structures, endocrine system, exocrine glands, female and male genitalia, germ cells, hemic/immune system, liver, musculoskeletal system, nervous system, pancreas, respiratory system, sense organs, skin, stomatognathic system, unclassified/mixed, and the urinary tract. For each category, the number of libraries in which the sequence was expressed were counted and shown over the total number of libraries in that category. Table 2 shows the expression of each polynucleotide, SEQ ID NOs: 1-9 in ovary, a tissue of the female genitalia category of the LIFESEQ GOLD database (Incyte Genomics). The first column shows library name; the second column, the number of cDNAs sequenced in that library; the third column, the description of the library; the fourth column, absolute abundance (Abund) of the transcript in the library; and the fifth column, percentage abundance (%Abund) of the transcript in the library.

TABLE 2
Transcript Images of Ovary Specific Polynucleotide Expression

| | | | | |
|---|---|---|---|---|
| Library | cDNAs | Description of Tissue | Abund | % Abund |

SEQ ID NO:1 (Incyte ID 329439)

| | | | | |
|---|---|---|---|---|
| OVARTUP08 | 1091 | ovary tumor, serous papillary CA, F, 3′CGAP | 5 | 0.4583 |
| OVARTUP05 | 2666 | ovary tumor, serous papillary carcinoma, F, 3′CGAP | 6 | 0.2251 |
| OVARTUP10 | 2162 | ovary tumor, carcinoma, borderline, F, 3′CGAP | 2 | 0.0925 |
| OVARTUP07 | 1136 | ovary tumor, serous papillary CA, F, 3′CGAP | 1 | 0.0880 |

SEQ ID NO:2 (Incyte ID 332630)

| | | | | |
|---|---|---|---|---|
| OVARTUP02 | 3144 | ovary tumor, serous papillary adenoCA, F, 3′CGAP | 2 | 0.0636 |
| OVARTUM02 | 2932 | ovary tumor, serous papillary CA, 64F, WM/WN | 1 | 0.0341 |

SEQ ID NO:3 (Incyte ID 396896)

| | | | | |
|---|---|---|---|---|
| OVARTUP08 | 1091 | ovary tumor, serous papillary CA, F, 3′CGAP | 2 | 0.1833 |
| OVARTUP05 | 2666 | ovary tumor, serous papillary carcinoma, F, 3′CGAP | 2 | 0.0750 |

SEQ ID NO:4 (Incyte ID 396924)

| | | | | |
|---|---|---|---|---|
| OVARTUP05 | 2666 | ovary tumor, serous papillary carcinoma, F, 3′CGAP | 5 | 0.1875 |
| OVARTUP07 | 1136 | ovary tumor, serous papillary CA, F, 3′CGAP | 2 | 0.1761 |
| OVARTUP08 | 1091 | ovary tumor, serous papillary CA, F, 3′CGAP | 1 | 0.0917 |

SEQ ID NO:5 (Incyte ID 403055)

| | | | | |
|---|---|---|---|---|
| OVARTUP09 | 709 | ovary tumor, carcinoma, borderline, F, 3′CGAP | 3 | 0.4231 |
| OVARTUP05 | 2666 | ovary tumor, serous papillary carcinoma, F, 3′CGAP | 9 | 0.3376 |
| OVARTUP08 | 1091 | ovary tumor, serous papillary CA, F, 3′CGAP | 2 | 0.1833 |
| OVARTUP07 | 1136 | ovary tumor, serous papillary CA, F, 3′CGAP | 1 | 0.0880 |
| OVARTUP10 | 2162 | ovary tumor, carcinoma, borderline, F, 3′CGAP | 1 | 0.0463 |

SEQ ID NO:6 (Incyte ID 441565)

| | | | | |
|---|---|---|---|---|
| OVARTUP05 | 2666 | ovary tumor, serous papillary carcinoma, F, 3′CGAP | 2 | 0.0750 |
| OVARTUP10 | 2162 | ovary tumor, carcinoma, borderline, F, 3′CGAP | 1 | 0.0463 |
| OVARTUP07 | 1136 | ovary tumor, serous papillary CA, F, 3′CGAP | 1 | 0.0880 |

SEQ ID NO:7 (Incyte ID 441710)

| | | | | |
|---|---|---|---|---|
| OVARTUP08 | 1091 | ovary tumor, serous papillary CA, F, 3′CGAP | 5 | 0.4583 |
| OVARTUP09 | 709 | ovary tumor, carcinoma, borderline, F, 3′CGAP | 3 | 0.4231 |
| OVARTUP10 | 2162 | ovary tumor, carcinoma, borderline, F, 3′CGAP | 8 | 0.3700 |
| OVARTUP05 | 2666 | ovary tumor, serous papillary carcinoma, F, 3′CGAP | 1 | 0.0375 |

SEQ ID NO:8 (Incyte ID 442177)

| | | | | |
|---|---|---|---|---|
| OVARTUP12 | 337 | ovary tumor, serous papillary CA, F, CGAP | 1 | 0.2967 |
| OVARTUP09 | 709 | ovary tumor, carcinoma, borderline, F, 3′CGAP | 1 | 0.1410 |
| OVARTUP10 | 2162 | ovary tumor, carcinoma, borderline, F, 3′CGAP | 2 | 0.0925 |
| OVARTUP08 | 1091 | ovary tumor, serous papillary CA, F, 3′CGAP | 1 | 0.0917 |
| OVARTUP07 | 1136 | ovary tumor, serous papillary CA, F, 3′CGAP | 1 | 0.0880 |

SEQ ID NO:9 (Incyte ID 1398162.1)

| | | | | |
|---|---|---|---|---|
| OVARTUP08 | 1096 | ovary tumor, serous papillary CA, F, 3′CGAP | 16 | 1.4599 |
| OVARTUP07 | 978 | ovary tumor, serous papillary CA, F, 3′CGAP | 9 | 0.9202 |
| OVARTUP05 | 1839 | ovary tumor, serous papillary carcinoma, F, 3′CGAP | 7 | 0.3806 |
| OVARTUP10 | 810 | ovary tumor, carcinoma, borderline, F, 3′CGAP | 1 | 0.1235 |
| OVARTUT10 | 1136 | ovary tumor, met colon adenoCA, 58F | 1 | 0.0277 |

*All mixed, pooled, normalized, and subtracted libraries have been removed from the table. Diseases attributed to mixed or pooled samples cannot be considered specific as to source, and the relative expression patterns of the polynucleotide in such libraries

cannot be considered specific. Normalized, subtracted or enriched libraries, that have had high copy number sequences removed before processing, are skewed to better represent low copy number sequences.

The transcript image clearly supports the use and conclusions of the method described in the DESCRIPTION OF THE INVENTION and demonstrates the expression of SEQ ID NOs: 1-9 in ovarian cancer, particularly serous papillary carcinoma.

VI Homology Searching of Polynucleotides and Their Deduced Proteins or Peptides

The polynucleotides of the Sequence Listing or their deduced amino acid sequences were used to query databases such as GenBank, SwissProt, BLOCKS, and the like. These databases that contain previously identified and annotated sequences or domains were searched using BLAST or BLAST 2 (Altschul et al. supra; Altschul, supra) to produce alignments and to determine which sequences were exact matches or homologs. The alignments were to sequences of prokaryotic (bacterial) or eukaryotic (animal, fungal, or plant) origin. Alternatively, algorithms such as the one described in Smith and Smith (1992, Protein Engineering 5:35-51) could have been used to deal with primary sequence patterns and secondary structure gap penalties. All of the sequences disclosed in this application have lengths of at least 49 nucleotides, and no more than 12% uncalled bases (where N is recorded rather than A, C, G, or T).

As detailed in Karlin (supra), BLAST matches between a query sequence and a database sequence were evaluated statistically and only reported when they satisfied the threshold of 10-25 for nucleotides and 10⁻¹⁴ for peptides. Homology was also evaluated by product score calculated as follows: the % nucleotide or amino acid identity [between the query and reference sequences] in BLAST is multiplied by the % maximum possible BLAST score [based on the lengths of query and reference sequences] and then divided by 100. In comparison with hybridization procedures used in the laboratory, the electronic stringency for an exact match was set at 70, and the conservative lower limit for an exact match was set at approximately 40 (with 1-2% error due to uncalled bases).

The BLAST software suite, freely available sequence comparison algorithms (NCBI, Bethesda, Md.), includes various sequence analysis programs including “blastn” that is used to align nucleic acid molecules and BLAST 2 that is used for direct pairwise comparison of either nucleic or amino acid molecules. BLAST programs are commonly used with gap and other parameters set to default settings, for example: Matrix: BLOSUM62; Reward for match: 1; Penalty for mismatch: −2; Open Gap: 5 and Extension Gap: 2 penalties; Gap×drop-off: 50; Expect: 10; Word Size: 11; and Filter: on. Identity or similarity is measured over the entire length of a sequence or some smaller portion thereof. Brenner et al. (1998; Proc Natl Acad Sci 95:6073-6078, incorporated herein by reference) analyzed the BLAST for its ability to identify structural homologs by sequence identity and found 30% identity is a reliable threshold for sequence alignments of at least 150 residues and 40%, for alignments of at least 70 residues.

The polynucleotides of this application were compared with assembled consensus sequences or templates found in the LIFESEQ GOLD database. Component sequences from polynucleotide, extension, full length, and shotgun sequencing projects were subjected to PHRED analysis and assigned a quality score. All sequences with an acceptable quality score were subjected to various pre-processing and editing pathways to remove low quality 3′ ends, vector and linker sequences, polyA tails, Alu repeats, mitochondrial and ribosomal sequences, and bacterial contamination sequences. Edited sequences had to be at least 50 bp in length, and low-information sequences and repetitive elements such as dinucleotide repeats, Alu repeats, and the like, were replaced by “Ns” or masked.

Edited sequences were subjected to assembly procedures in which the sequences were assigned to gene bins. Each sequence could only belong to one bin, and sequences in each bin were assembled to produce a template. Newly sequenced components were added to existing bins using BLAST and CROSSMATCH. To be added to a bin, the component sequences had to have a BLAST quality score greater than or equal to 150 and an alignment of at least 82% local identity. The sequences in each bin were assembled using PHRAP. Bins with several overlapping component sequences were assembled using DEEP PHRAP. The orientation of each template was determined based on the number and orientation of its component sequences.

Bins were compared to one another and those having local similarity of at least 82% were combined and reassembled. Bins having templates with less than 95% local identity were split. Templates were subjected to analysis by STITCHER/EXON MAPPER algorithms (Incyte Genomics) that analyze the probabilities of the presence of splice variants, alternatively spliced exons, splice junctions, differential expression of alternative spliced genes across tissue types or disease states, and the like. Assembly procedures were repeated periodically, and templates were annotated using BLAST against GenBank databases such as GBpri. An exact match was defined as having from 95% local identity over 200 base pairs through 100% local identity over 100 base pairs and a homolog match as having an E-value (or probability score) of ≦1×10⁻⁸. The templates were also subjected to frameshift FASTx against GENPEPT, and homolog match was defined as having an E-value of ≦1×10⁻⁸. Template analysis and assembly was described in U.S. Ser. No. 09/276,534, filed Mar. 25, 1999.

Following assembly, templates were subjected to BLAST, motif, and other functional analyses and categorized in protein hierarchies using methods described in U.S. Ser. No. 08/812,290 and U.S. Ser. No. 08/811,758, both filed Mar. 6, 1997; in U.S. Ser. No. 08/947,845, filed Oct. 9, 1997; and in U.S. Ser. No. 09/034,807, filed Mar. 4, 1998. Then templates were analyzed by translating each template in all three forward reading frames and searching each translation against the PFAM database of hidden Markov model-based protein families and domains using the HMMER software package (Washington University School of Medicine, St. Louis, Mo.).

The polynucleotide was further analyzed using MACDNASIS PRO software (Hitachi Software Engineering, San Francisco, Calif.), and LASERGENE software (DNASTAR) and queried against public databases such as the GenBank rodent, mammalian, vertebrate, prokaryote, and eukaryote databases, SwissProt, BLOCKS, PRINTS, PFAM, and Prosite.

VII Hybridization Technologies and Analyses

Immobilization of Polynucleotides on a Substrate

The polynucleotides are applied to a substrate by one of the following methods. A mixture of polynucleotides is fractionated by gel electrophoresis and transferred to a nylon membrane by capillary transfer. Alternatively, the polynucleotides are individually ligated to a vector and inserted into bacterial host cells to form a library. The polynucleotides are then arranged on a substrate by one of the following methods. In the first method, bacterial cells containing individual clones are robotically picked and arranged on a nylon membrane. The membrane is placed on LB agar containing selective agent (carbenicillin, kanamycin, ampicillin, or chloramphenicol depending on the vector used) and incubated at 37C. for 16 hr. The membrane is removed from the agar and consecutively placed colony side up in 10% SDS, denaturing solution (1.5 M NaCl, 0.5 M NaOH), neutralizing solution (1.5 M NaCl, 1 M Tris-HCl, pH 8.0), and twice in 2×SSC for 10 min each. The membrane is then UV irradiated in a STRATALINKER UV-crosslinker (Stratagene).

In the second method, polynucleotides are amplified from bacterial vectors by thirty cycles of PCR using primers complementary to vector sequences flanking the insert. PCR amplification increases a starting concentration of 1-2 ng nucleic acid to a final quantity greater than 5 μg. Amplified nucleic acids from about 400 bp to about 5000 bp in length are purified using SEPHACRYL-400 beads (APB). Purified nucleic acids are arranged on a nylon membrane manually or using a dot/slot blotting manifold and suction device and are immobilized by denaturation, neutralization, and UV irradiation as described above. Purified nucleic acids are robotically arranged and immobilized on polymer-coated glass slides using the procedure described in U.S. Pat. No. 5,807,522. Polymer-coated slides are prepared by cleaning glass microscope slides (Corning, Acton, Mass.) by ultrasound in 0.1% SDS and acetone, etching in 4% hydrofluoric acid (VWR Scientific Products, West Chester, Pa.), coating with 0.05% aminopropyl silane (Sigma-Aldrich) in 95% ethanol, and curing in a 110C. oven. The slides are washed extensively with distilled water between and after treatments. The nucleic acids are arranged on the slide and then immobilized by exposing the array to UV irradiation using a STRATALINKER UV-crosslinker (Stratagene). Arrays are then washed at room temperature in 0.2% SDS and rinsed three times in distilled water. Non-specific binding sites are blocked by incubation of arrays in 0.2% casein in phosphate buffered saline (PBS; Tropix, Bedford, Mass.) for 30 min at 60C.; then the arrays are washed in 0.2% SDS and rinsed in distilled water as before.

Probe Preparation for Membrane Hybridization

Hybridization probes derived from the polynucleotides of the Sequence Listing are employed for screening cDNAs, mRNAs, or genomic DNA in membrane-based hybridizations. Probes are prepared by diluting the polynucleotides to a concentration of 40-50 ng in 45 μl TE buffer, denaturing by heating to 100C. for five min, and briefly centrifuging. The denatured polynucleotide is then added to a REDIPRIME tube (APB), gently mixed until blue color is evenly distributed, and briefly centrifuged. Five μl of [³²P]dCTP is added to the tube, and the contents are incubated at 37C. for 10 min. The labeling reaction is stopped by adding 5 μl of 0.2M EDTA, and probe is purified from unincorporated nucleotides using a PROBEQUANT G-50 microcolumn (APB). The purified probe is heated to 100C. for five min, snap cooled for two min on ice, and used in membrane-based hybridizations as described below.

Probe Preparation for Polymer Coated Slide Hybridization

Hybridization probes derived from mRNA isolated from samples are employed for screening polynucleotides of the Sequence Listing in array-based hybridizations. Probe is prepared using the GEMbright kit (Incyte Genomics) by diluting mRNA to a concentration of 200 ng in 9 μl TE buffer and adding 5 μl 5×buffer, 1 μl 0.1 M DTT, 3 μl Cy3 or Cy5 labeling mix, 1 μl RNAse inhibitor, 1 μl reverse transcriptase, and 5 μl 1×yeast control mRNAs. Yeast control mRNAs are synthesized by in vitro transcription from noncoding yeast genomic DNA (W. Lei, unpublished). As quantitative controls, one set of control mRNAs at 0.002 ng, 0.02 ng, 0.2 ng, and 2 ng are diluted into reverse transcription reaction mixture at ratios of 1:100,000, 1:10,000, 1:1000, and 1:100 (w/w) to sample mRNA respectively. To examine mRNA differential expression patterns, a second set of control mRNAs are diluted into reverse transcription reaction mixture at ratios of 1:3, 3:1, 1:10, 10:1, 1:25, and 25:1 (w/w). The reaction mixture is mixed and incubated at 37C. for two hr. The reaction mixture is then incubated for 20 min at 85C., and probes are purified using two successive CHROMASPIN+TE 30 columns (Clontech, Palo Alto. Calif.). Purified probe is ethanol precipitated by diluting probe to 90 μl in DEPC-treated water, adding 2 μl 1 mg/ml glycogen, 60 μl 5 M sodium acetate, and 300 μl 100% ethanol. The probe is centrifuged for 20 min at 20,800×g, and the pellet is resuspended in 12 μl resuspension buffer, heated to 65C. for five min, and mixed thoroughly. The probe is heated and mixed as before and then stored on ice. Probe is used in high density array-based hybridizations as described below.

Membrane-based Hybridization

Membranes are pre-hybridized in hybridization solution containing 1% Sarkosyl and 1×high phosphate buffer (0.5 M NaCl, 0.1 M Na₂HPO₄, 5 mM EDTA, pH 7) at 55C for two hr. The probe, diluted in 15 ml fresh hybridization solution, is then added to the membrane. The membrane is hybridized with the probe at 55C. for 16 hr. Following hybridization, the membrane is washed for 15 min at 25C. in 1 mM Tris (pH 8.0), 1% Sarkosyl, and four times for 15 min each at 25C. in 1 mM Tris (pH 8.0). To detect hybridization complexes, XOMAT-AR film (Eastman Kodak, Rochester, N.Y.) is exposed to the membrane overnight at −70C., developed, and examined visually.

Polymer Coated Slide-based Hybridization

Probe is heated to 65C. for five min, centrifuged five min at 9400 rpm in a 5415C. microcentrifuge (Eppendorf Scientific, Westbury, N.Y.), and then 18 μl are aliquoted onto the array surface and covered with a coverslip. The arrays are transferred to a waterproof chamber having a cavity just slightly larger than a microscope slide. The chamber is kept at 100% humidity internally by the addition of 140 μl of 5×SSC in a corner of the chamber. The chamber containing the arrays is incubated for about 6.5 hr at 60C. The arrays are washed for 10 min at 45C. in 1×SSC, 0.1% SDS, and three times for 10 min each at 45C. in 0.1×SSC, and dried.

Hybridization reactions are performed in absolute or differential hybridization formats. In the absolute hybridization format, probe from one sample is hybridized to array elements, and signals are defected after hybridization complexes form. Signal strength correlates with probe mRNA levels in the sample. In the differential hybridization format, differential expression of a set of polynucleotides in two biological samples is analyzed. Probes from the two samples are prepared and labeled with different labeling moieties. A mixture of the two labeled probes is hybridized to the array elements, and signals are examined under conditions in which the emissions from the two different labels are individually detectable. Elements on the array that are hybridized to substantially equal numbers of probes derived from both biological samples give a distinct combined fluorescence (Shalon WO95/35505).

Hybridization complexes are detected with a microscope equipped with an INNOVA 70 mixed gas 10 W laser (Coherent, Santa Clara, Calif.) capable of generating spectral lines at 488 nm for excitation of Cy3 and at 632 nm for excitation of Cy5. The excitation laser light is focused on the array using a 20X microscope objective (Nikon, Melville, N.Y.). The slide containing the array is placed on a computer-controlled X-Y stage on the microscope and raster-scanned past the objective with a resolution of 20 micrometers. In the differential hybridization format, the two fluorophores are sequentially excited by the laser. Emitted light is split, based on wavelength, into two photomultiplier tube detectors (PMT R1477, Hamamatsu Photonics Systems, Bridgewater, N.J.) corresponding to the two fluorophores. Appropriate filters positioned between the array and the photomultiplier tubes are used to filter the signals. The emission maxima of the fluorophores used are 565 nm for Cy3 and 650 nm for CyS. The sensitivity of the scans is calibrated using the signal intensity generated by the yeast control mRNAs added to the probe mix. A specific location on the array contains a complementary DNA sequence, allowing the intensity of the signal at that location to be correlated with a weight ratio of hybridizing species of 1:100,000.

The output of the photomultiplier tube is digitized using a 12-bit RTI-835H analog-to-digital (A/D) conversion board (Analog Devices, Norwood, Mass.) installed in an IBM-compatible PC computer. The digitized data are displayed as an image where the signal intensity is mapped using a linear 20-color transformation to a pseudocolor scale ranging from blue (low signal) to red (high signal). The data is also analyzed quantitatively. Where two different fluorophores are excited and measured simultaneously, the data are first corrected for optical crosstalk (due to overlapping emission spectra) between the fluorophores using the emission spectrum for each fluorophore. A grid is superimposed over the fluorescence signal image such that the signal from each spot is centered in each element of the grid. The fluorescence signal within each element is then integrated to obtain a numerical value corresponding to the average intensity of the signal. The software used for signal analysis is the GEMTOOLS program (Incyte Genomics).

VIII Complementary Molecules

Molecules complementary to the polynucleotide, from about 5 (PNA) to about 5000 bp (complement of an entire cDNA insert), are used to detect or inhibit gene expression. These molecules are selected using LASERGENE software (DNASTAR). Detection is described in Example VII. To inhibit transcription by preventing promoter binding, the complementary molecule is designed to bind to the most unique 5′ sequence and includes nucleotides of the 5′ UTR upstream of the initiation codon of the open reading frame. Complementary molecules include genomic sequences (such as enhancers or introns) and are used in “triple helix” base pairing to compromise the ability of the double helix to open sufficiently for the binding of polymerases, transcription factors, or regulatory molecules. To inhibit translation, a complementary molecule is designed to prevent ribosomal binding to the mRNA encoding the protein.

Complementary molecules are placed in expression vectors and used to transform a cell line to test efficacy; into an organ, tumor, synovial cavity, or the vascular system for transient or short term therapy; or into a stem cell, zygote, or other reproducing lineage for long term or stable gene therapy. Transient expression lasts for a month or more with a non-replicating vector and for three months or more if appropriate elements for inducing vector replication are used in the transformation/expression system.

Stable transformation of appropriate dividing cells with a vector encoding the complementary molecule produces a transgenic cell line, tissue, or organism (U.S. Pat. No. 4,736,866). Those cells that assimilate and replicate sufficient quantities of the vector to allow stable integration also produce enough complementary molecules to compromise or entirely eliminate activity of the polynucleotide encoding the protein.

IX Protein Expression

Expression and purification of the protein are achieved using either a cell expression system or an insect cell expression system. The pUB6N5-His vector system (Invitrogen, Carlsbad, Calif.) is used to express protein in CHO cells. The vector contains the selectable bsd gene, multiple cloning sites, the promoter/enhancer sequence from the human ubiquitin C gene, a C-terminal V5 epitope for antibody detection with anti-V5 antibodies, and a C-terminal polyhistidine (6×His) sequence for rapid purification on PROBOND resin (Invitrogen). Transformed cells are selected on media containing blasticidin.

Spodoptera frugiperda (Sf9) insect cells are infected with recombinant Autographica californica nuclear polyhedrosis virus (baculovirus). The polyhedrin gene is replaced with the cDNA by homologous recombination and the polyhedrin promoter drives cDNA transcription. The protein is synthesized as a fusion protein with 6×his which enables purification as described above. Purified protein is used in the following activity and to make antibodies

X Production of Antibodies

The protein is purified using polyacrylamide gel electrophoresis and used to immunize mice or rabbits. Antibodies are produced using the protocols below. Alternatively, the amino acid sequence of the expressed protein is analyzed using LASERGENE software (DNASTAR) to determine regions of high antigenicity. An antigenic epitope, usually found near the C-terminus or in a hydrophilic region is selected, synthesized, and used to raise antibodies. Typically, epitopes of about 15 residues in length are produced using a 431A peptide synthesizer (ABI) using Fmoc-chemistry and coupled to KLH (Sigma-Aldrich) by reaction with N-maleimidobenzoyl-N-hydroxysuccinimide ester to increase antigenicity.

Rabbits are immunized with the epitope-KLH complex in complete Freund's adjuvant. Immunizations are repeated at intervals thereafter in incomplete Freund's adjuvant. After a minimum of seven weeks for mouse or twelve weeks for rabbit, antisera are drawn and tested for antipeptide activity. Testing involves binding the peptide to plastic, blocking with 1% bovine serum albumin, reacting with rabbit antisera, washing, and reacting with radio-iodinated goat anti-rabbit IgG. Methods well known in the art are used to determine antibody titer and the amount of complex formation.

XI Purification of Naturally Occurring Protein Using Specific Antibodies

Naturally occurring or recombinant protein is purified by immunoaffinity chromatography using antibodies which specifically bind the protein. An immunoaffinity column is constructed by covalently coupling the antibody to CNBr-activated SEPHAROSE resin (APB). Media containing the protein is passed over the immunoaffinity column, and the column is washed using high ionic strength buffers in the presence of detergent to allow preferential absorbance of the protein. After coupling, the protein is eluted from the column using a buffer of pH 2-3 or a high concentration of urea or thiocyanate ion to disrupt antibody/protein binding, and the protein is collected.

XII Screening Molecules for Specific Binding with the Polynucleotide or Protein

The polynucleotide or the protein are labeled with ³²P-dCTP, Cy3-dCTP, or Cy5-dCTP (APB), or with BIODIPY or FITC (Molecular Probes, Eugene, Oreg.), respectively. Libraries of candidate molecules or compounds previously arranged on a substrate are incubated in the presence of labeled polynucleotide or protein. After incubation under conditions for either a nucleic acid or amino acid sequence, the substrate is washed, and any position on the substrate retaining label, which indicates specific binding or complex formation, is assayed, and the ligand is identified. Data obtained using different concentrations of the nucleic acid or protein are used to calculate affinity between the labeled nucleic acid or protein and the bound molecule.

XIII Two-Hybrid Screen

A yeast two-hybrid system, MATCHMAKER LexA Two-Hybrid system (Clontech Laboratories, Palo Alto, Calif.), is used to screen for peptides that bind the protein of the invention. A polynucleotide encoding the protein is inserted into the multiple cloning site of a pLexA vector, ligated, and transformed into E. coli. A cDNA, prepared from mRNA, is inserted into the multiple cloning site of a pB42AD vector, ligated, and transformed into E. coli to construct a cDNA library. The pLexA plasmid and pB42AD-cDNA library constructs are isolated from E. coli and used in a 2:1 ratio to co-transform competent yeast EGY48[p8op-lacZ] cells using a polyethylene glycol/lithium acetate protocol. Transformed yeast cells are plated on synthetic dropout (SD) media lacking histidine (−His), tryptophan (−Trp), and uracil (−Ura), and incubated at 30C. until the colonies have grown up and are counted. The colonies are pooled in a minimal volume of 1×TE (pH 7.5), replated on SD/−His/−Leu/−Trp/−Ura media supplemented with 2% galactose (Gal), 1% raffinose (Raf), and 80 mg/ml 5-bromo-4-chloro-3-indolyl β-d-galactopyranoside (X-Gal), and subsequently examined for growth of blue colonies. Interaction between expressed protein and cDNA fusion proteins activates expression of a LEU2 reporter gene in EGY48 and produces colony growth on media lacking leucine (−Leu). Interaction also activates expression of β-galactosidase from the p8op-lacZ reporter construct that produces blue color in colonies grown on X-Gal.

Positive interactions between expressed protein and cDNA fusion proteins are verified by isolating individual positive colonies and growing them in SD/−Trp/−Ura liquid medium for 1 to 2 days at 30C. A sample of the culture is plated on SD/−Trp/−Ura media and incubated at 30C. until colonies appear. The sample is replica-plated on SD/−Trp/−Ura and SD/−His/−Trp/−Ura plates. Colonies that grow on SD containing histidine but not on media lacking histidine have lost the pLexA plasmid. Histidine-requiring colonies are grown on SD/Gal/Raf/X-Gal/−Trp/−Ura, and white colonies are isolated and propagated. The pB42AD-cDNA plasmid, which contains a polynucleotide encoding a protein that physically interacts with the protein, is isolated from the yeast cells and characterized.

All patents and publications mentioned in the specification are incorporated by reference herein. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the field of molecular biology or related fields are intended to be within the scope of the following claims.

What is claimed is:

1. A combination comprising a plurality of polynucleotides wherein the plurality of polynucleotides have the nucleic acid sequences of SEQ ID NOs: 1-9 or the complements thereof.

2. An isolated polynucleotide comprising a nucleic acid sequence selected from SEQ ID NOs: 1-9 and the complements thereof.

3. A method of using a combination to screen a plurality of molecules to identify at least one ligand which specifically binds a polynucleotide of the combination, the method comprising:
a) contacting the combination of claim 1 with molecules under conditions to allow specific binding; and
b) detecting specific binding, thereby identifying a ligand which specifically binds the polynucleotide.

4. The method of claim 3 wherein the plurality of molecules or compounds are selected from DNA molecules, peptides, peptide nucleic acid molecules, proteins, repressors, RNA molecules, and transcription factors.

5. A method for using a combination to detect expression in a sample containing nucleic acids, the method comprising:
a) hybridizing the combination of claim 1 to the nucleic acids under conditions for formation of one or more hybridization complexes; and
b) detecting hybridization complex formation, wherein complex formation indicates expression in the sample.

6. The method of claim 5 wherein the polynucleotides of the combination are attached to a substrate.

7. The method of claim 5 wherein the sample is ovarian tissue.

8. The method of claim 5 wherein the nucleic acids of the sample are amplified prior to hybridization.

9. The method of claim 5 wherein the comparison with standards is diagnostic of an ovarian cancer.

10. A composition comprising a polynucleotide of claim 2.

11. A vector comprising a polynucleotide of claim 2.

12. A host cell comprising the vector of claim 11.

13. A method for using a host cell to produce a protein, the method comprising:
a) culturing the host cell of claim 12 under conditions for expression of the protein; and
b) recovering the protein from cell culture.

14. A purified protein comprising a polypeptide produced by the method of claim 13.

15. A composition comprising the protein of claim 14.

16. A method for using a protein to screen a plurality of molecules to identify at least one ligand which specifically binds the protein, the method comprising:
a) combining the protein of claim 14 with the plurality of molecules under conditions to allow specific binding; and
b) detecting specific binding, thereby identifying a ligand which specifically binds the protein.

17. The method of claim 16 wherein the plurality of molecules is selected from agonists, antagonists, antibodies, DNA molecules, peptides, peptide nucleic acids, proteins, and RNA molecules.

18. A method of using a protein to screen a plurality of antibodies to identify an antibody which specifically binds the protein, the method comprising:
a) contacting a plurality of antibodies with the protein of claim 14 under conditions to form an antibody:protein complex, and
b) dissociating the antibody from the antibody:protein complex, thereby obtaining antibody which specifically binds the protein.

19. A method for preparing a polyclonal antibody, the method comprising:
a) immunizing a animal with protein of claim 14 under conditions to elicit an antibody response,
b) isolating animal antibodies,
c) attaching the protein to a substrate,
d) contacting the substrate with isolated antibodies under conditions to allow specific binding to the protein, and
e) dissociating the antibodies from the protein, thereby obtaining purified polyclonal antibodies.

20. An antibody which specifically binds a protein produced by the method of claim 18.


SEQUENCE LISTING (9 sequences)

SEQ ID NO: 1 - DNA, 257 bp, Homo sapiens
  misc_feature: Incyte ID No 329439.1
gcgagctgctattttttcctgcaatgcactgttctttggtttgggaaatntnctatatttntaccgtgctttaaanatacacaatggttctaaataactacttttctttaaanttanatgtaacatcttaattaaaatgtnatccataaatnaggnacagtctgtgaggttgtncgagcgtgaaagctccacagtctgaggcctggagaccccttctgtcgtcttctcgcaagccgtatagtagtagtagaggccgc

SEQ ID NO: 2 - DNA, 1066 bp, Homo sapiens
  misc_feature: Incyte ID No 332630.1
attttctgctgaccagtttgccttctattttatgggctcagtattccttacctgcctcttcccatgctaaagatggcccaccttcgttttgttatttaagcaacttcatcccctggcttgttttcacaagtgggtttnctgagcctttgactctaagtcatctaattgaacattgtgttgtgatataaaaagtaagttaggcttgtgtttttcaccaggccatttcattgtatcctaactaggtgggtggctgtgataaatgtacagattagccaatacagaatcacgtctaattccaagttttcttttgggtatgaagttgagacatggggaagcttgagctttgttttgtcagcaacaggtgaggtggagaactgggactgaaggggtttggggaggatctgtttaagattggaaaaaatacatcaacttgggaatgtaagcaactagaaccaagcaatctgtacaacgtttttactgttggctgtcttcctctgggaaactaaaagccattttgttgatagcacttcaggtcagaattcatcaacagggaatggaaacattgtttatatgctttggggtacatcaagataagttgagggtcaagttaatgtcatgccacaatcaaccctgtatgtcagggcccttgagagcagagtagtgtcgggagagtgggtggtatggttgacacaaagacccacaggtatttttatgggttcacttaatgaagcaggggcttagttgagggtagagactgttcattaaaccagcctttgttgaccactccctgcagtatggacaggacatgatcaccatccttaagtcccactatagcaggggggaaacagtatgcttaattaagcttaattataaactctttgagttagaaaactggtgaaagtgttatttcctcctgaagtaattatgtatatatacatgccaattccaatcagaatgctaatttttctttttaaccccagagctgtgcaaaatgtttctcaaatttattcaggaacataaacaacagttggaaaggacaagaactgtctccagcataatggttaag

SEQ ID NO: 3 - DNA, 198 bp, Homo sapiens
  misc_feature: Incyte ID No 396896.1ext
gcctactactactactatacggctgcgagaagacgacagaagggaagtggtaagggacagggaaggaaaggacagaaaacacaaaacaaaacaaaacaaaacaaaacaaaacaaaacaaaatgattacaaaactcatctgcagatgaacacaattaacctaaaaaaaaaaaaaaaaaaaaaaaaaaagtcgtatcgat

SEQ ID NO: 4 - DNA, 343 bp, Homo sapiens
  misc_feature: Incyte ID No 396924.1
tgcctggtcaggtgtctttgagagtggtgctatcccactgaactggtgagaaagttgagtagaaccaaagaaagagaacttgatagaagcaagaattatgattgcgaatcactagcccttctgattttctccagtctaaattatgttttgctcattttacttgaccagtaaggtaaaaccccacgtgggaggaaggggagttgctgtttcataatgggtcagaatcaaaaccccattgtcccaagccaagctttcagacaggctgactccctgcatttttctaagtcaaaataaaaacaattcccttctgtcgtcttctcgcaacagtatagtagtagtaggc

SEQ ID NO: 5 - DNA, 316 bp, Homo sapiens
  misc_feature: Incyte ID No 403055.1ext
aattcttaagaaaagcagtgtgccagggcctgcccctcacacttggaagtgacccaggaggtgctgcgtgctgcctcactgggtctcactccagccgcgctttgctcctctctgttcttgcacttgcctcagtggcctctgcagcagagcctcatgccagctcttccctcttcttgggatgcccctgttatttttccctgtagtcttggagggccggctccttggtcattattcacatgtcatctctgtgccacctcctagactgctgacttgcccccacagcaacccccttctgtcgtcttctcgcagccgtata

SEQ ID NO: 6 - DNA, 172 bp, Homo sapiens
  misc_feature: Incyte ID No 441565.1
gcctagtatgaaaatatacccaataccaccttctttattgctgactgggaatgtcctctcaaagctcctaaaattcttgactgtctccttttttgcctttctctagctggactattttgattatacccttctgtcatcttctcgcagccgtatagtagtagtaggcggccgc

SEQ ID NO: 7 - DNA, 179 bp, Homo sapiens
  misc_feature: Incyte ID No 441710.1ext
tttttggcatttaacaatcagatcccaaaatgtctttcctgactggctcccaccgcttctctggactgttccaggaccctgactagtgcatgcactctgtaaggtgcttgtgctggtccctcctcttgatagcccttctgtcgtcttctcgcagccgtatagtagtagtaggcggccgc

SEQ ID NO: 8 - DNA, 127 bp, Homo sapiens
  misc_feature: Incyte ID No 442177.1
gagcccttctcaggcagaggaggtcaggcaggtacacgtgcctttgggaagaaggtggtggaaaaatatggaataatgagcccttctgtcgtcttctcgcagccgtatagtagtagtaggcgaccgc

SEQ ID NO: 9 - DNA, 3900 bp, Homo sapiens
  misc_feature: Incyte ID No 1398162.1
gcgagcggcggcacgacgaggggaaaagagctgagcgagaccaaagtcagccgggagacagtgggtctgtgagagaccgaatagaggggctggggccacgagcgccattgacaagcaatggggaagaaacagaaaaacaagagcgaagacagcaccaaggatgacattgatcttgatgccttggctgcagaaatagaaggagctggtgctgccaaagaacaggagcctcaaaagtcaaaagggaaaaagaaaaaagagaaaaaaaagcaggactttgatgaagatgatatcctgaaagaactggaagaattgtctttggaagctcaaggcatcaaagctgacagagaaactgttgcagtgaagccaacagaaaacaatgaagaggaattcacctcaaaagataaaaaaaagaaaggacagaagggcaaaaaacagagttttgatgataatgatagcgaagaattggaagataaagattcaaaatcaaaaaagactgcaaaaccgaaagtggaaatgtactctgggagtgatgatgatgatgattttaacaaacttcctaaaaaagctaaagggaaagctcaaaaatcaaataagaagtgggatgggtcagaggaggatgaggataacagtaaaaaaattaaagagcgttcaagaatgaattcttctggtgaaagtggtgatgaatcagatgaatttttgcaatctagaaaaggacagaaaaaaaatcagaaaaacaagccaggtcctaacatagaaagtgggaatgaagatgatgacgcctccttcaaaattaagacagtggcccaaaagaaggcagaaaagaaggagcgcgagagaaaaaagcgagatgaagaaaaagcgaaactgcggaagctgaaagaaagagaagagttagaaacaggtaaaaaggatcagagtaaacaaaaggaatctcaaaggaaatttgaagaagaaactgtaaaatccaaagtgactgttgatactggagtaattcctgcctctgaagagaaagcagagactcccacagctgcagaagatgacaatgaaggagacaaaaagaagaaagataagaagaaaaagaaaggagaaaaggaagaaaaagagaaagagaagaaaaaaggacctagcaaagccactgttaaagctatgcaagaagctctggctaagcttaaagaggaagaagaaagacagaagagagaagaggaagaacgtataaaacggcttgaagaattagaagccaagcgtaaagaagaggaacgattggaacaagaaaaaagagaaaggaaaaagcaaaaagaaaaagaaagaaaagaacgcttgaaaaaagaagggaaacttttaactaaatcccagagagaagccagagccagagccgaagctactcttaaactgctacaagctcagggtgttgaagtgccatcaaaagactctttgccaaagaagaggccaatttatgaagataaaaagaggaaaaaaataccacagcagctagaaagtaaagaagtgtctgaatcaatggaattatgtgctgctgtagaagttatggaacaaggagtaccagaaaaggaagagacaccacctcctgttgaaccagaagaagaagaagatactgaggatgctggattggatgattgggaagctatggccagtgatgaggagacagaaaaagtagaaggaaacacagttcatatagaagtaaaagaaaaccctgaagaggaggaggaggaggaagaagaggaagaagaagatgaagaaagtgaagtagaggaggaagaggagggagaaagtgaaggcagtgaaggtgatgaggaagatgaaaaggtgtcagatgagaaggattcagggaagacattagataaaaagccaagtaaagaaatgagctcagattctgaatatgactctgatgatgatcggactaaagaagaaagggcttatgacaaagcaaaacggaggattgagaaacggcgacttgaacatagtaaaaatgtaaacaccgaaaagctaagagcccctattatctgcgtacttgggcatgtggacacagggaagacaaaaattctagataagctccgtcacacacatgtacaagacggtgaagcaggtggtatcacacaacaaatttgggccaccaatgttcctcttgaagctattaatgaacagactaagatgattaaaaattttgatagagagaatgtacggattccaggaatgctaattattgatactcctgggcatgaatctttcagtaatctgagaaatagaggaagctctctttgtgacattgccattttagttgttgatattatgcatggtttggagccccagacaattgagtctatcaaccttctcaaatctaaaaaatgtcccttcattgttgcactcaataagattgataggttatatgattggaaaaagagtcctgactctgatgtggctgctactttaaagaagcagaaaaagaatacaaaagatgaatttgaggagcgagcaaaggctattattgtagaatttgcacagcagggtttgaatgctgctttgttttatgagaataaagatccccgcacttttgtgtctttggtacctacctctgcacatactggtgatggcatgggaagtctgatctaccttcttgtagagttaactcagaccatgttgagcaagagacttgcacactgtgaagagctgagagcacaggtgatggaggttaaagctctcccggggatgggcaccactatagatgtcattttgatcaatgggcgtttgaaggaaggagatacaatcattgttcctggagtagaagggcccattgtaactcagattcgaggcctcctgttacctcctcctatgaaggaattacgagtgaagaaccagtatgaaaagcataaagaagtagaagcagctcagggggtaaagattcttggaaaagacctggagaaaacattggctggtttacccctccttgtggcttataaagaagatgaaatccctgttcttaaagatgaattgatccatgagttaaagcagacactaaatgctatcaaattagaagaaaaaggagtctatgtccaggcatctacactgggttctttggaagctctactggaatttctgaaaacatcagaagtgccctatgcaggaattaacattggcccagtgcataaaaaagatgttatgaaggcttcagtgatgttggaacatgaccctcagtatgcagtaattttggccttcgatgtgagaattgaacgagatgcacaagaaatggctgatagtttaggagttagaatttttagtgcagaaattatttatcatttatttgatgcctttacaaaatatagacaagactacaagaaacagaaacaagaagaatttaagcacatagcagtatttccctgcaagataaaaatcctccctcagtacatttttaattctcgagatccgatagtgatgggggtgacggtggaagcaggtcaggtgaaacaggggacacccatgtgtgtcccaagcaaaaattttgttgacatcggaatagtaacaagtattgaaataaaccataaacaagtggatgttgcaaaaaaaggacaagaagtttgtgtaaaaatagaacctatccctggtgagtcacccaaaatgtttggaagacattttgaagctacagatattcttgttagtaagatcagccggcagtccattgatgcactcaaagactggttcagagatgaaatgcagaagagtgactggcagcttattgtggagctgaagaaagtatttgaaatcatctaattttttcacatggagcaggaactggagtaaatgcaatactgtgttgtaatatcccaacaaaaatcagacaaaaaatggaacagacgtatttggacactgatggacttaagtatggaagga